(19) United States
(12) Patent Application Publication
(10) Pub. No.: US 2020/0390764 A1
(43) Pub. Date: Dec. 17, 2020

(54) PHARMACEUTICAL FORMULATIONS, PROCESSES FOR PREPARATION, AND METHODS OF USE

(71) Applicant: Sunesis Pharmaceuticals, Inc., South San Francisco, CA (US)

(72) Inventor: Gene Curtis JAMIESON, Boulder Creek, CA (US)

(21) Appl. No.: 16/859,111

(22) Filed: Apr. 27, 2020

Related U.S. Application Data
(63) Continuation of application No. 16/185,793, filed on Nov. 9, 2018, now abandoned.
(60) Provisional application No. 62/583,891, filed on Nov. 9, 2017.

(51) Int. Cl. A61K 31/4985; A61K 31/496; A61K 47/32; A61K 47/10; A61K 47/12; A61K 9/10; A61P 35/00; A61K 47/38; C07D 487/04; C07D 471/04; A61K 45/06; A61K 31/497

(52) U.S. Cl. CPC A61K 31/4985; A61K 31/496; A61K 47/32; A61K 47/10; A61K 47/12; A61K 9/10; A61K 9/4858; A61K 47/38; C07D 487/04; C07D 471/04; A61K 45/06; A61K 31/497; A61P 35/00

(57) ABSTRACT

The invention relates to pharmaceutical compositions, comprising a solid dispersion extrudate comprising any of certain active compounds that modulate cellular survival pathways implicating certain protein kinases, as described, for the treatment of cancer, and processes for the preparation of such compositions. The invention also relates to methods of administering such pharmaceutical compositions to patients for the treatment of cancer.

### RELATED APPLICATIONS

This application is a continuation of U.S. application Ser. No. 16/185,793, filed on Nov. 9, 2018, which claims priority to and the benefit of U.S. Provisional Application No. 62/583,891, filed on Nov. 9, 2017, the entire contents of each of which are incorporated herein by reference in their entireties.

### BACKGROUND

The 3-phosphoinositide-dependent protein kinase-1 (PDK1, also known as PDPK1) is a master kinase that activates other kinases important in cell growth and survival including members of the Akt (protein kinase B, PKB), protein kinase C (PKC), p90 ribosomal S6 kinase RSK (S6K), and SGK families. PDK1 activates substrate kinases via activation T-loop phosphorylation (Belham et al., Curr. Biol., 1999, 9:R93-R96).

PDK1 is a 556-amino acid protein that consists of an N-terminal kinase (catalytic) domain, and a C-terminal pleckstrin homology (PH) domain. The PH domain interacts with phosphatidylinositol (PI) (3,4)-bisphosphate and phosphatidylinositol (3,4,5)-trisphosphate, contributing to localization and activation of certain PDK1 substrates, notably including Akt. The activation of Akt is believed to require a proper orientation of the kinase and PH domains of PDK1 and Akt at the membrane. Akt is itself known to be associated with cancers (Manning et al., Cell, 2007, 129(7):1261-1274), and is frequently mutated or hyperactivated in human cancers.

However, while PDK1 can interact with certain of its substrates through this PI-dependent (PH-mediated) mechanism, it can interact with other substrates through a distinct PI-independent mechanism. The N-terminal kinase domain has three ligand binding sites; a substrate binding site, an ATP binding site, and a docking site (also known as PIF pocket) for interaction with substrates. This docking site is known as the “PIF pocket,” referring to its binding to a region of protein kinase C-related kinase-2 (PRK2), termed the PDK1-interacting fragment (PIF) (Biondi et al., EMBO J., 2000, 19(5):979-988). Several PDK1 substrates, including S6K and PKC, require binding at this PIF pocket docking site.

As noted, PDK1 is important in regulating the activity of other kinases. Principal targets of PDK1 are the AGC subfamily of protein kinases (Alessi et al., Biochem. Soc. Trans, 2001, 29(2):1-14), such as isoforms of protein kinase B (PKB, also known as Akt), p70 ribosomal S6 kinase (S6K) (Avruch et al., Prog. Mol. Subcell. Biol., 2001, 26:115), p90 ribosomal S6 kinases (RSK1-4) (Frodin et al., EMBO J., 2000, 19:2924-2934), IKK and members of the protein kinase C (PKC) family (Le Good et al., Science, 1998, 281:2042-2045). PDK1-mediated signaling increases in response to insulin, growth factors, and extracellular matrix cell binding (integrin signaling) resulting in diverse cellular events such as cell survival, growth, proliferation, and glucose regulation (Lawlor et al., J. Cell Sci., 2001, 114:2903-2910; Lawlor et al., EMBO J., 2002, 21:3728-3738). Of the several PDK1 substrates mentioned above, much attention has focused on AKT. Development of potent and selective AKT inhibitors has been challenging and only two compounds have made it into clinical development: AZD5363 and MK2206. These compounds have shown promising anti-cancer activity in certain tumor types. However, more recent studies using these compounds have revealed, surprisingly, that many tumor types are not sensitive to AKT inhibition or express no or little activated AKT.

PDK1 is the only kinase known to phosphorylate Thr308 in the activation loop of AKT that is critical for activation of AKT kinase. Thus, PDK1 plays a critical role in AKT activation. Efforts to develop potent and selective PDK1 inhibitors with suitable drug like properties have been unsuccessful and no compounds have entered clinical development. Reported pre-clinical studies with PDK1 inhibitors GSK2334470 and BX-320/-795 have shown moderate efficacy and thus, it has been proposed that PDK1 may not be rate limiting in promoting cancer cell growth. Alternatively, these inhibitors may simply have poor pharmacological properties, failing to achieve sufficient inhibition to produce an effect, or the type of cancers cells used did not depend on PDK1 for growth.

The tumor-suppressor phosphatase with tensin homology (PTEN) is an important negative regulator of the cell-survival signaling pathway initiated by phosphatidylinositol 3-kinase (PI3K). The PDK1/Akt pathway is activated in many cancers via mutations in Receptor Tyrosine Kinases (RTKs), Ras, PI-3 kinase, or PTEN (Cully et al., Nature Reviews Cancer, 2006, 6:184-192). Elevated PDK1 activation and signaling has been detected in several cancers resulting from distinct genetic events such as PTEN mutations or over-expression of certain key regulatory proteins (Graff, Expert Opin. Ther. Targets, 2002, 6:103-113, Brognard et al., Cancer Res., 2001, 61:3986-3997). In fact, PTEN is one of the most frequently mutated genes in human cancer. PDK1 has been found to be overexpressed in acute myeloid leukemia (Zabkiewicz et al., Haematologica, 2014, 99(5):858-864). The potential of PDK1 inhibitors as anti-cancer compounds was indicated by transfection of a PTEN negative human cancer cell line (U87MG) with antisense oligonucleotides directed against PDK1. The resulting decrease in PDK1 protein levels led to a reduction in cellular proliferation and survival (Flynn et al., Curr. Biol., 2000, 10:1439-1442).

RSK2 (p90RSK2) is one of four ribosomal S6 kinases (S6K) known in humans, a family of serine/threonine kinases that are activated by the MAPK/ERK pathway. RSK comprises two kinase domains: the C-terminal domain autophosphorylates RSK2, which is necessary for its activation; the N-terminal domain of activated RSK2 phosphorylates downstream substrates such as certain transcriptional regulators. It is possible that RSK2 plays a key role in tumors that are not dependent on AKT or provides a key resistance mechanism to bypass AKT signaling upon treatment with AKT inhibitors.

RSK2 is known to be activated through phosphorylation by PDK1 through the PI-independent, PIF pocket mechanism, and promotes cellular proliferation in various cell types, and may contribute to certain cancers. For example, RSK2 has been shown to be activated in certain forms of myeloid leukemia. Inhibition of RSK2 induced apoptotic cell death in Molm14 and Mv(4; 11) leukemia cells and primary samples from AML patients, but failed to affect apoptosis in Ba/F3 or K562 cells or in primary samples from CML patients (Elf et al., Blood, 2011, 117(25):6885-6894). Separately, it has been reported that RSK2 inhibition induced apoptosis in certain myeloma cells, and that receptor tyrosine kinase fibroblast growth factor receptor 3 (FGFR3) activates RSK2, which may induce hematopoietic transformation (Kang et al., J. Biol. Chem., 2008, 283(8):4652-4657; Kang et al., Mol. Cell. Biol., 2009, 29(8):2105-2117).

Compounds that inhibit the activity of PDK1 are described, for example, in international patent applications WO 2008/005457 A2 and WO 2011/044157 A1. International patent application WO 2017/070565 A1 discloses compounds that impair or block PI-independent, PIF pocket-mediated substrate binding and have broad anti-tumor activity in hematologic cancers and other cancers. On the one hand, it has been found that these compounds appear to modify the conformation of PDK1 to block PIF binding, thereby preventing the binding and phosphorylation of PI-independent (PIF-dependent) substrates, while yet inhibiting PDK1 kinase activity by also blocking ATP binding. This dual-mechanism function may by critical to effectively inhibit PDK1 signaling by affecting both PI-dependent and PI-independent substrate phosphorylation. This function, therefore, could make these compounds useful in treatment of cancers that are Akt-independent or in which resistance to Akt inhibitors arises. In addition, such dual-mechanism inhibitors may have utility in treatment of cancers that are dependent for growth on RSK2 activity or other PIF-dependent substrates downstream of PDK1, whether or not AKT is active.

The advancement of such dual-mechanism inhibitors has been somewhat hampered by physical characteristics of some of the compounds, specifically their pharmaceutical properties such as bioavailability in the context of oral administration. For example, some of these compounds have low aqueous solubility and moderate log p. Both parameters can adversely affect oral bioavailability. Any improvement in the physical characteristics of these compounds would potentially offer a more beneficial therapy.

Accordingly, it is an object of the present invention to provide pharmaceutical compositions comprising dual-mechanism inhibitor compounds that are stable and that allow for rapid dissolution and enhanced oral bioavailability. Furthermore, it is believed that the efficacy of these compounds correlates with drug exposure. Accordingly, it is desirable to be able to administer such compounds at the highest possible dose, i.e., the highest possible dose at which the side-effect profile is acceptable. A dosing regimen that achieves a higher exposure to the compounds thereby would provide a meaningful benefit in the treatment of patients suffering from cancer.

### SUMMARY OF THE INVENTION

The present invention provides pharmaceutical compositions as described herein with superior properties, including rapid dissolution and increased oral bioavailability. The present invention also provides processes for the preparation of said pharmaceutical compositions. Furthermore, the present invention provides an intermittent dosing regimen for the improved treatment of cancer.

Accordingly, the present invention relates to the following:

1) A pharmaceutical composition comprising (a) a solid dispersion extrudate comprising a compound described herein or a pharmaceutically acceptable salt thereof and (b) one or more pharmaceutically acceptable excipients.
2) A process for preparing a pharmaceutical composition, which comprises the steps of:
(i) extruding a mixture comprising a compound described herein or a pharmaceutically acceptable salt thereof and a polymer carrier (e.g., vinylpyrrolidinone-vinyl acetate copolymer), a solubilizer/plasticizer (e.g., PEG 1500, and a bioavailability enhancer (e.g., d-α-tocopheryl polyethylene glycol 1000 succinate (TPGS), to form a solid dispersion extrudate;
(ii) blending the resulting solid dispersion extrudate with one or more pharmaceutically acceptable excipients.
   3) A method for the treatment of cancer in a patient in need of such treatment, comprising administering an effective amount of a pharmaceutical composition comprising a compound described herein or a pharmaceutically acceptable salt thereof and one or more pharmaceutically acceptable excipients, to the patient according to an intermittent dosing regimen, in which the dosing regimen comprises administering the composition once or twice a week and the total amount of the compound administered each week is about 400 mg to about 1,000 mg.

The present invention provides a process for the preparation of a pharmaceutical composition comprising active compounds as described herein, with improved absorption. For example, Compound 1 exhibits a low solubility (0.1 mg/mL) and a moderate c Log p (5.34), thus the bioavailability of Compound 1 is limited by its solubility. We have found that the dissolution property of the active compounds can be improved by making amorphous solid dispersions prepared by hot melt extrusion. According to the process of the present invention, it is possible to provide, from an active compound, a formulation in which the dissolution rate and oral bioavailability of the drug are high. Furthermore, the solid dispersion extrudates of the present invention have good stability at room temperature.

The pharmaceutical composition of the present invention has superior effects as a medicament in cancers in which the growth, proliferation, or survival of the cancer is mediated at least in part by PDK1 activity. The pharmaceutical composition of the present invention can be administered orally and safely to a patient.

The present invention provides a method for the treatment of cancer in a patient, in which the cancer is characterized by PDK1 activity, by intermittent administration of a pharmaceutical composition as described here, in which the intermittent dosing regimen is a weekly administration and the amount administered each week is about 400 mg to about 1,000 mg. The intermittent dosing regimen provides a higher unit dose, which allows for the achievement of higher concentrations of the active compound and a higher degree of pathway inhibition for a window of time within the dosing interval, without compromising overall dose density.

It is believed, without being bound by theory, that clinical benefits afforded by the pharmaceutical compositions disclosed herein will result from improved bioavailability and higher exposures of the incorporated active compound.

Pharmaceutical compositions according to the invention employ active compounds selected from:

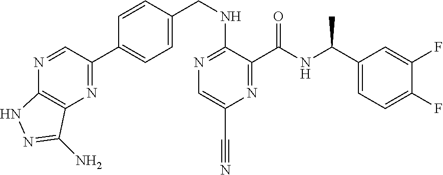 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide (Compound 1),

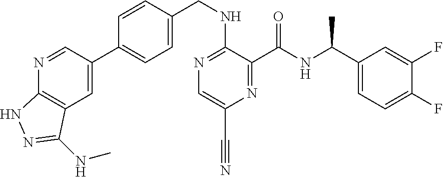 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide (Compound 2), and

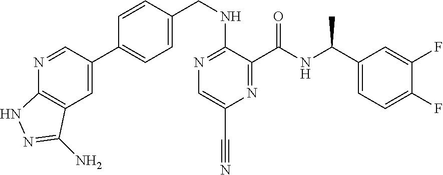 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide (Compound 3),
   and pharmaceutically acceptable salts of any of the foregoing. Such compounds are useful as modulators of cellular survival pathways implicating certain protein kinases (e.g., PDK1, RSK2, Akt), and thus are useful, for example, for the treatment of PDK1-, RSK2-, and Akt-mediated diseases.

In certain embodiments, the invention provides pharmaceutical compositions comprising solid dispersion extrudate comprising an active compound as described herein, in which the compound is present in an amount effective to inhibit a PDK1-PIF mediated substrate interaction-dependent cancer survival pathway, such as an RSK2-dependent pathway, or an Akt-independent pathway, that is implicated in cancer growth and survival. In certain other embodiments, the invention provides pharmaceutical compositions comprising an active compound as described herein and optionally further comprising an additional therapeutic agent. In yet other embodiments, the additional therapeutic agent is an agent for the treatment of cancer.

In yet another aspect, the present invention provides methods for inhibiting a kinase activation pathway implicated in cancer growth and survival in a patient or a biological sample, comprising administering to said patient an effective inhibitory amount of a pharmaceutical composition comprising a solid dispersion extrudate comprising an active compound as described herein. In still another aspect, the present invention provides methods for treating any disorder involving such a kinase activation pathway, comprising administering to a subject in need thereof a therapeutically effective amount of a pharmaceutical composition comprising a solid dispersion extrudate comprising an active compound as described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### BRIEF DESCRIPTION OF THE DRAWINGS

FIG. 1 shows pharmacokinetic (PK) results as concentration versus time for three different formulations of Compound 1 measured using a liquid oral dosage administered to rats. Black circle=Compound 1 (10% w/w) nano-milling suspension. Red triangle=lipid-based liquid formulation of Compound 1 (10% w/w), PEG400 (85% w/w), TPGS (2.5% w/w), PEG 1500 (2.5% w/w), DMSO (5% w/w), and NMP (5% w/w). Blue square=Compound 1 (10% w/w) solution in PEG 400.

FIG. 2 shows pharmacokinetic results as concentration versus time for three different formulations of Compound 1 measured using a solid oral dosage form administered to dogs. Red circle=HME of 10% Compound 1 and 90% of Kollidon® VA64. Blue triangle=HME of 10% Compound 1, 60% Kollidon® VA64, 20% PEG 1500, and 10% TPGS. Black square=Compound 1 (10% w/w) solution in PEG 400.

FIG. 3 shows results of kinetic dissolution of solid dispersion extrudates of Compound 1 in copovidone or HPMCAS as concentration versus time. “Cmpd 1” is Compound 1 APi as a reference. 1-04A=10% Compound 1/10% PEG 1500/10% TPGS/70% VA-64 at 130° C. 1-04B=10% Compound 1/10% PEG 1500/10% TPGS/70% VA-64 at 140° C. 1-04C=10% Compound 1/10% PEG 1500/10% TPGS/70% VA-64 at 150° C. 1-05A=10% Compound 1/10% TPGS/80% HPMCAS (MF) at 130° C. 1-05B=10% Compound 1/10% TPGS/80% HPMCAS (MF) at 140° C. 1-05C=10% Compound 1/10% TPGS/80% HPMCAS (MF) at 150° C.

FIG. 4 shows results of kinetic dissolution of solid dispersion extrudates of Compound 2 in copovidone or HPMCAS as concentration versus time. “Cmpd 2” is Compound 2 as a reference. 2-02A=10% Compound 2/10% PEG 1500/10% TPGS/70% VA-64 at 130° C. 2-02B=10% Compound 2/10% PEG 1500/10% TPGS/70% VA-64 at 140° C. 2.02C=10% Compound 2/10% PEG 1500/10% TPGS/70% VA-64 at 150° C.

FIG. 5 shows results of kinetic dissolution of solid dispersion extrudates having varying amounts of Compound 1 in TPGS/PEG 1500/copovidone as concentration versus time. “Cmpd 1” is Compound 1 as a reference. 1−06=10% Compound 1/10% PEG 1500/10% TPGS/70% VA-64 at 150° C. 1−09=20% Compound 1/10% PEG 1500/10% TPGS/60% VA-64 at 150° C. 1−10=30% Compound 1/10% PEG 1500/10% TPGS/50% VA-64 at 150° C.

FIG. 6 shows results of kinetic dissolution of solid dispersion extrudates having varying amounts of Compound 2 in TPGS/PEG 1500/copovidone as concentration versus time. “Cmpd 2” is Compound 2 as a reference. 2-02C=10% Compound 2/10% PEG 1500/10% TPGS/70% VA-64 at 150° C. 2−07=20% Compound 2/10% PEG 1500/10% TPGS/60% VA-64 at 150° C. 2-08=130% Compound 2/10% PEG 1500/10% TPGS/50% VA-64 at 150° C.

### DETAILED DESCRIPTION OF CERTAIN EMBODIMENTS

### Compounds Useful in Methods of the Invention

PDK1 can interact with its substrates through phosphatidyl-inositol (PI)-dependent (PH-mediated) or PI-independent (PIF-mediated) mechanisms. Active compounds as described herein, as described below, are believed to occupy both the ATP-binding pocket and the adaptive (“allosteric”) pocket and block PI-independent substrate binding and have anti-tumor activity in solid tumors and hematologic cancers. Such active compounds as described herein have a distinct activity profile, which manifests in the ability to impair the growth, proliferation, or survival of cancer cells, such as cells that are resistant to Akt inhibition, that are resistant to inhibition of PDK1 catalytic activity, or that are dependent on RSK2 activity.

Thus, in one aspect, the present invention provides methods of use of active compounds selected from:

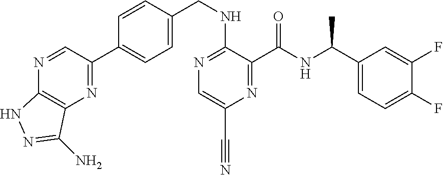 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide (Compound 1),

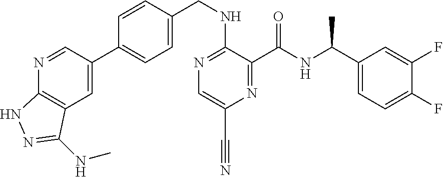 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide (Compound 2), and

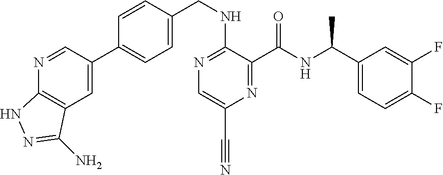 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide (Compound 3),
   and pharmaceutically acceptable salts of any of the foregoing.

For example, active compounds as described herein may be used to inhibit the growth, proliferation, or survival of cancer cells in which PDK1-PIF-mediated substrate interaction-dependent cell survival pathways are implicated.

In some embodiments, the invention provides a method of treating cancer in a subject in need thereof by inducing cancer cell apoptosis through inhibition of PDK1-PIF mediated substrate interaction-dependent cancer survival pathways, comprising administering to said subject a therapeutically effective amount of an active compound as described herein.

In some embodiments, the invention provides a method of treating cancer in a subject in need thereof by inhibiting PDK1-PIF mediated substrate interaction-dependent cancer cell growth, proliferation, or survival, comprising administering to said subject a therapeutically effective amount of an active compound as described herein.

In some embodiments, the invention provides a method for inhibiting the growth, proliferation, or survival of cancer cells by inhibiting Akt-independent cancer cell growth, proliferation, or survival pathways dependent on PDK1-PIF mediated substrate interaction, the method comprising contacting the cancer cells with an effective amount of an active compound as described herein.

In some embodiments, the invention provides a method for inducing apoptosis of cancer cells by inhibiting Akt-independent cancer cell survival pathways dependent on PDK1-PIF mediated substrate interaction, the method comprising contacting the cancer cells with an effective amount of an active compound as described herein.

In some embodiments, the invention provides a method of inhibiting the growth, proliferation, or survival of cancer cells, the growth, proliferation, or survival of which is dependent on PIF-mediated substrate binding by PDK1, the method comprising contacting the cancer cells with an active compound as described herein in an amount sufficient to inhibit growth, proliferation, or survival of the cancer cells.

In some embodiments, the invention provides a method of inducing apoptosis of cancer cells, the growth, proliferation, or survival of which is dependent on PIF-mediated substrate binding by PDK1, the method comprising contacting the cancer cells with an effective amount of an active compound as described herein.

In some embodiments, the invention provides a method of inhibiting PIF-mediated substrate binding by PDK1 in cancer cells, comprising contacting the cells with an active compound as described herein, whereby growth, proliferation, or survival of the cancer cells is inhibited.

In some embodiments, the invention provides a method of inducing apoptosis in cancer cells, comprising contacting cancer cells with an active compound as described herein that inhibits PIF-mediated substrate binding by PDK1.

In some embodiments, the invention provides a method of preparing a medicament for use in the treatment of cancer in which the growth, proliferation, or survival of the cancer is dependent on a PDK1-PIF-mediated substrate interaction, comprising a therapeutically effective amount of an active compound as described herein and a pharmaceutically acceptable excipient.

In some embodiments, the invention provides a product comprising a container and a medicament for use in the treatment of cancer in which the growth, proliferation, or survival of the cancer is dependent on a PDK1-PIF-mediated substrate interaction, in which the medicament comprises an active compound as described herein and a pharmaceutically acceptable excipient.

In another aspect, active compounds as described herein may be used to inhibit the growth, proliferation, or survival of cancer cells in which RSK2-dependent cell survival pathways are implicated.

In some embodiments, the invention provides a method of treating cancer in a subject in need thereof by inducing cancer cell apoptosis through inhibition of RSK2-dependent survival pathways, comprising administering to said subject a therapeutically effective amount of an active compound as described herein.

In some embodiments, the invention provides a method of treating cancer in a subject in need thereof by inhibiting RSK2-dependent cancer cell growth, proliferation, or survival, comprising administering to said subject a therapeutically effective amount of an active compound as described herein.

In some embodiments, the invention provides a method of inhibiting the growth, proliferation, or survival of cancer cells, the growth, proliferation, or survival of which is dependent on kinase activity of RSK2, the method comprising contacting the cancer cells with an active compound as described herein in an amount sufficient to inhibit RSK2 activity in the cancer cells.

In some embodiments, the invention provides a method of inducing apoptosis in cancer cells, comprising contacting cancer cells with an active compound as described herein that inhibits RSK2 activation by PDK1.

In another aspect, active compounds described herein may be used to inhibit the growth, proliferation, or survival of cancer cells in which Akt-independent cell survival pathways are implicated. Such cells are considered to be resistant to inhibition of Akt. Thus, cells that can survive, or that are resistant to, or do not respond to, Akt inhibitors, may yet be inhibited by active compounds as described herein.

In some embodiments, the invention provides a method of treating cancer in a patient in need thereof by inducing cancer cell apoptosis through inhibition of Akt-independent cancer cell survival pathways, comprising administering to said subject a therapeutically effective amount of an active compound as described herein.

In some embodiments, the invention provides a method of treating cancer in a subject in need thereof by inhibiting Akt-independent cancer cell growth, proliferation, or survival, comprising administering to said subject a therapeutically effective amount of an active compound as described herein.

In some embodiments, the invention provides a method of inhibiting the growth, proliferation, or survival of cancer cells, the growth, proliferation, or survival of which is not dependent on kinase activity of Akt, the method comprising contacting the cancer cells with an active compound as described herein in an amount sufficient to inhibit growth, proliferation, or survival of the cancer cells.

In some embodiments, the invention provides a method of inducing apoptosis of cancer cells, the growth, proliferation, or survival of which is not dependent on kinase activity of Akt, the method comprising contacting the cancer cells with an effective amount of an active compound as described herein.

In some embodiments, the invention provides a method of inhibiting the growth, proliferation, or survival of cancer cells, the growth, proliferation, or survival of which is dependent on kinase activity of Akt, the method comprising contacting the cancer cells with an active compound as described herein in an amount sufficient to inhibit growth, proliferation, or survival of the cancer cells.

In some embodiments, the invention provides a method of inhibiting the growth, proliferation, or survival of cancer cells, the growth, proliferation, or survival of which is dependent on kinase activity of Akt, the method comprising contacting the cancer cells with an active compound as described herein in an amount sufficient to inhibit growth, proliferation, or survival of the cancer cells with an effective amount of an active compound as described herein.

In some embodiments, the invention provides a method of inducing apoptosis in cancer cells in which viability is Akt-independent, comprising contacting the cancer cells with an amount of an active compound as described herein that is effective to interfere with PIF-mediated substrate binding by PDK1 in the cancer cells.

In some embodiments, the invention provides a method of inhibiting Akt-independent growth, proliferation, or survival of cancer cells, comprising contacting the cancer cells with an effective amount of an active compound as described herein.

In some embodiments, the invention provides a method treating a patient having a cancer, the growth, proliferation, or survival of which is Akt-independent, comprising administering to the subject an amount of an active compound as described herein that is effective to impair growth, proliferation, or survival of the cancer.

In some embodiments, the invention provides a method of inducing apoptosis of cancer cells, the growth, proliferation, or survival of which is dependent on PDK1 PIF-binding activity, the method comprising contacting the cancer cells with an effective amount of an active compound as described herein.

In some embodiments, the invention provides a method of inducing apoptosis of cancer cells, the growth, proliferation, or survival of which is dependent on RSK2 activity, the method comprising contacting the cancer cells with an effective amount of an active compound as described herein.

Such compounds and methods of their preparation are described in detail in international patent publications WO 2011-044157 A1 and WO 2017/070565 A1, the entire contents of which are incorporated herein by reference.

In certain embodiments, the active compound used in methods of the invention is:

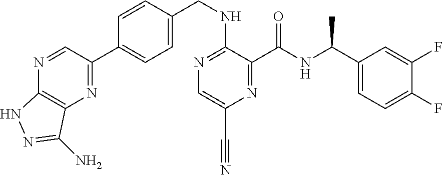 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide, or a pharmaceutically acceptable salt thereof.

In certain embodiments, the active compound used in methods of the invention is:

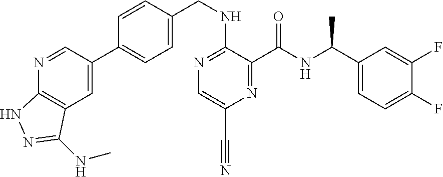 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, or a pharmaceutically acceptable salt thereof.

In certain embodiments, the active compound used in the methods of the invention is:

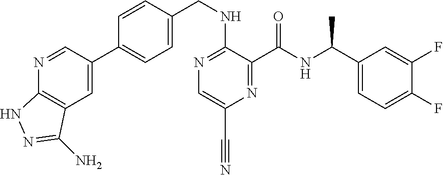 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide, or a pharmaceutically acceptable salt thereof.

In another aspect, the invention provides a use of an active compound as described herein for the preparation of a medicament for the treatment of cancer in which PDK1-PIF-mediated substrate interaction-dependent cell survival pathways are implicated.

In another aspect, the invention provides a use of an active compound as described herein for the preparation of a medicament for the treatment of cancer in which RSK2-dependent cell survival pathways are implicated.

In another aspect, the invention provides a use of an active compound as described herein for the preparation of a medicament for the treatment of cancer in which Akt-independent cell survival pathways are implicated.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where one enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L., Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972).

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

As used herein, “effective amount” means an amount of a therapeutic substance (e.g., a composition of the invention) that is (1) sufficient upon appropriate administration to a patient (a) to cause a detectable decrease in the severity of the disorder or disease state being treated; (b) to ameliorate or alleviate the patient's symptoms of the disease or disorder; or (c) to slow or prevent advancement of: or otherwise stabilize or prolong stabilization of, the disorder or disease state being treated (e. g., prevent additional tumor growth of a cancer); and (2) equal to or less than the maximum tolerated dose (MTD). In any form or composition, the clinically effective amount can be expressed as amount of therapeutic substance per patient BSA, e.g., as mg/m2.

As used herein, “patient” means a human being diagnosed with, exhibiting symptoms of or otherwise believed to be afflicted with or suffering from a disease, disorder, or condition.

The terms “a,” “an,” “them” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein.

As used herein, the illustrative terms “including,” “such as,” “for example,” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means “including but not limited to.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. Unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Consistent with guidance from US FDA regarding naming of drug products, an “active moiety” means the molecule or ion, excluding those appended portions of the molecule that cause a compound to be a salt (including a salt with hydrogen or coordination bonds), or other noncovalent derivative (such as a complex, chelate, or clathrate) of the molecule. The active moiety is responsible for the physiological or pharmacological action of the drug substance without regard to the actual charged state of the molecule in vivo. For example, the active moiety of a hydrochloride salt of a base is the free base and not the protonated form of the base. The active moiety of a metal salt of an acid is the free acid.

When an active ingredient occurs as a salt, the ingredient will be expressed, unless otherwise indicated, using the name of the active moiety and not the name of the salt (e.g., “Compound A” rather than “Compound A hydrochloride”). The strength of a pharmaceutical product will also be expressed in terms of the active moiety (e.g., “100 mg Compound A”) rather than the salt strength equivalent (i.e., “123.7 mg Compound A hydrochloride”). Likewise, a dose to be administered to a patient will be expressed in terms of the active moiety rather than the salt. A unit dose, too, will be expressed in terms of the active moiety rather than the salt.

Thus, when an amount of an active ingredient in a solid dispersion extrudate of the invention is indicated, the amount will be understood as an amount of the physical form employed, which may be a salt or other derivative. In general, processes of manufacture will reference the active ingredient rather than the active moiety. By contrast, an amount identified in a pharmaceutical product (incorporating a pharmaceutical composition comprising a solid dispersion extrudate of an active compound and one or more other excipients) will be understood as an amount of the active moiety. Similarly, an amount indicated for use in a method of treatment will be understood as an amount of the active moiety.

In another aspect, active compounds as described herein are useful for the treatment of one or more diseases, disorders, and/or conditions that may be alleviated by inhibiting (i.e. decreasing) certain PDK1 activities, including PI-independent PIF pocket substrate binding and PDK1-PIF mediated substrate interaction-dependent cell growth, proliferation, or survival. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In one aspect, the present invention provides methods of treating cancer in a subject in need thereof. In some embodiments, provided methods include administering to the subject a therapeutically effective amount of a provided compound. The term “cancer” includes diseases or disorders involving abnormal cell growth and/or proliferation. In some embodiments, a cancer treated in accordance with the present invention is, by way of nonlimiting example, glioma, thyroid carcinoma, breast carcinoma, lung cancer (e.g., small-cell lung carcinoma, non-small-cell lung carcinoma), gastric carcinoma, cervical carcinoma, melanoma, skin carcinoma, colorectal carcinoma, gastrointestinal stromal tumors, pancreatic carcinoma, bile duct carcinoma, ovarian carcinoma, endometrial carcinoma, prostate carcinoma, renal cell carcinoma, anaplastic large-cell lymphoma, leukemia (e.g., acute myeloid leukemia, T-cell leukemia, chronic lymphocytic leukemia), multiple myeloma, malignant mesothelioma, malignant melanoma, colon cancer (e.g. microsatellite instability-high colorectal cancer).

In another aspect, the present invention provides methods of treating cancers that are hematologic cancers. In some embodiments, provided methods include administering to the subject a therapeutically effective amount of a provided compound. The term “hematologic cancer” includes blood-borne tumors and diseases or disorders involving abnormal cell growth and/or proliferation in tissues of hematopoietic origin, such as lymphomas, leukemias, and myelomas. Hematologic cancers that may be treated according to the invention include, by way of nonlimiting example, anaplastic large-cell lymphoma, non-Hodgkin's lymphoma, Hodgkin's lymphoma, B-cell lymphoma (e.g., ABC-diffuse large B-cell lymphoma, GCB-diffuse large B-cell lymphoma), T-cell lymphoma, mantle cell lymphoma, histiocytic lymphoma, T-cell leukemia, chronic lymphocytic leukemia, multiple myeloma, chronic myeloid leukemia, acute lymphocytic leukemia, acute myelogenous leukemia, and acute myeloblastic leukemia, plasma cell leukemia.

As used herein, the term “precancerous condition” means a condition, abnormal tissue growth, or lesion that tends or is likely to become cancerous. Precancerous conditions include, for example, actinic keratosis, adenomatous polyps of the colon, cervical dysplasia, and antecedent hematological disorders such as myelofibrosis, aplastic anemia, paroxysmal nocturnal hemoglobinuria, polycythemia vera, and myelodysplastic syndrome.

### Assays

To develop useful inhibitors of cancer growth, proliferation, or survival, candidate inhibitors capable of decreasing PDK1-PIF-mediated substrate interaction-dependent cell survival pathways may be identified in vitro. The activity of provided compounds can be assayed utilizing methods known in the art, such as, for example, those methods presented in international patent applications WO 2008/005457 A2, WO 2011/044157 A1, and WO 2017/070565 A1.

Compounds that decrease PDK1-PIF-mediated substrate interaction-dependent cell survival pathways may be identified and tested using biologically active PDK1 and other elements of these pathways, either recombinant or naturally-occurring. PDK1, RSK2, and Akt, for example, can be found in native cells, isolated in vitro, or co-expressed or expressed in a cell. Measuring the reduction in the PDK1-PIF-mediated substrate interaction-dependent cell survival pathways in the presence of an inhibitor relative to the activity in the absence of the inhibitor may be performed using a variety of methods known in the art, such as in the assays described herein. Other methods for assaying the activity of elements of PDK1-PIF-mediated substrate interaction-dependent cell survival pathways are known in the art. The selection of appropriate assay methods is well within the capabilities of those of skill in the art.

Compounds may be further tested in cell models or animal models for their ability to cause a detectable change in phenotype related to PDK1-PIF-mediated substrate interaction-dependent cell survival pathways. In addition to cell cultures, animal models may be used to test inhibitors of PDK1 for their ability to treat cancer in an animal model.

Compounds may be further tested for their ability to selectively inhibit or induce expression of genes or proteins that could serve as biomarkers to monitor inhibition of PDK1 activity in animal models or in healthy subjects or in patients.

### Pharmaceutical Compositions

In another aspect, the present invention provides pharmaceutical compositions comprising an active compound, optionally in combination with a pharmaceutically acceptable excipient (e.g., a carrier).

Provided pharmaceutical compositions include optical isomers, diastereomers, or pharmaceutically acceptable salts of the compounds disclosed herein. For example, in some embodiments, pharmaceutical compositions include a pharmaceutically acceptable salt. A compound included in the pharmaceutical composition may be covalently attached to a pharmaceutically acceptable carrier. Alternatively, the inventive compound included in the pharmaceutical composition is not covalently linked to a pharmaceutically acceptable carrier.

A “pharmaceutically acceptable carrier,” as used herein refers to pharmaceutical excipients, for example, pharmaceutically, physiologically, acceptable organic, or inorganic carrier substances suitable for enteral or parenteral application which do not deleteriously react with the compounds used in accordance with the provided methods. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds used in accordance with the provided methods.

As used herein, the term “substantially amorphous” refers to a solid material having little or no long range order in the position of its molecules. For example, a substantially amorphous material has less than about 30% crystallinity (e.g., less than about 25% crystallinity, less than about 20% crystallinity, less than about 15% crystallinity, less than about 10% crystallinity, less than about 5% crystallinity, less than about 4% crystallinity). It is also noted that the term ‘substantially amorphous’ materials include ‘amorphous’ materials, which refers to materials having no (0%) observable crystallinity.

As used herein, the term “crystalline” and related terms used herein, when used to describe a substance, component or product is substantially crystalline, as determined by X-ray diffraction, polarized optical microscopy and/or FT-Raman microscopy.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. A “pharmaceutically acceptable salt” means any non-toxic salt of an active compound that, upon administration to a recipient, is capable of providing, either directly or indirectly, that active compound or an active metabolite or residue thereof.

Pharmaceutically acceptable salts are well known in the art. For example, S. M. Berge et al., describe pharmaceutically acceptable salts in detail in J. Pharmaceutical Sciences, 1977, 66:1-19, incorporated herein by reference. Pharmaceutically acceptable salts of Compounds 1, 2, or 3 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C₁₋₄alkyl)₄ salts. This invention also envisions the quaternization of any basic nitrogen-containing groups of an active compound. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The pharmaceutical compositions of the invention include compositions solid dispersion extrudate is substantially amorphous. In one aspect, the substantially amorphous pharmaceutical composition comprises an amount of crystalline active compound, or a pharmaceutically acceptable salt thereof. In one aspect, the amount of crystalline active compound is less than about 30%, less than about 29%, less than about 28%, less than about 27%, less than about 26%, less than about 25%, less than about 20%, less than about 15%, less than about 10%, less than about 5%, or less than about 4%.

The substantially amorphous character of a solid dispersion extrudate can be detected using analytical methods, including but not limited to, microscopic methods (scanning electronic microscopy (SEM), polarized light microscopy (PLM), hot stage microscopy (HSM), thermal methods (differential scanning calorimetry (DSC) modulated DSC (rnDSC), diffraction methods (XRPD). and spectroscopic methods (FT-Infrared (IR), FT-Raman, solid state NMR (ssNMR), and confocal Raman microscopy (CRM). In one aspect, the amorphous character of a pharmaceutical composition is detected by X-ray powder diffraction (XRPD).

In one aspect, the amount of crystalline substance in a substantially amorphous pharmaceutical composition can be determined using a calibration curve based on samples of variable crystalline content (high and low regions). In one aspect, the amount of crystalline active compound in a substantially amorphous solid dispersion extrudcate of the invention may affect the solubility of the composition. In one aspect, the amount of crystalline active compound in a substantially amorphous solid dispersion extrudate of the invention may affect the bioavailability of the composition. In one aspect, less than about 30% of crystalline active compound in a substantially amorphous solid dispersion extrudate does not reduce the solubility and/or bioavailability of the composition. In another aspect, less than about 29%, less than about 28%, less than about 27%, less than about 26%, less than about 24%, less than about 25%, less than about 20%, less than about 15%, less than about 10%, less than about 5%, less than about 4% of crystalline active compound in a substantially amorphous solid dispersion extrudate does not significantly reduce the solubility and/or bioavailability of the composition.

The solid dispersion extrudate materials of the invention include a polymer carrier. The polymer carrier may be any substance that is suitable for use in hot melt extrusion processes described herein and compatible with the active compounds as described herein. For example, the polymer carrier may be a vinylpyrrolidone-vinyl acetate copolymer. One example of a vinylpyrrolidone-vinyl acetate copolymer is a copovidone. Copovidone materials are available commercially, such as the Kollidon® polymers from (Bayer), including, for example, Kollidon® VA64 (CAS 25086-89-9). Other copovidione materials that are freely water soluble may be used. For example, useful copovidone materials may be copolymers of 6 parts of N-vinylpyrrolidone and 4 parts of vinyl acetate. Such materials may have a molecular weight of about 45,000 g/mol. Materials with physicochemical properties similar to those of such copovidone materials (e.g., plasticity, solubilization, etc.) may be used as polymer carriers according to the invention. Blends of such materials may be used as the polymer carrier component of these extrudates.

The solid dispersion extrudate materials of the invention include a solubilizer. The solubilizer may be any substance that is suitable for use in hot melt extrusion processes describe herein and compatible with the active compounds as described herein. For example, the solubilizer may be a polyethylene glycol 1500 (PEG 1500; CAS 25322-68-3). Materials with physicochemical properties similar to those of such PEG 1500 materials may be used as solubilizers according to the invention. Blends of such materials may be used as the solubilizer component of these extrudates.

The solid dispersion extrudate materials of the invention include a bioavailability enhancer. The bioavailability enhancer may be any substance that is suitable for use in hot melt extrusion processes describe herein and compatible with the active compounds as described herein. For example, the bioavailability enhancer may be d-α-tocopheryl polyethylene glycol 1000 succinate (TPGS), an ester of esterification of the acid crystalline d-α-tocopheryl acid succinate with polyethylene glycol 1000, e.g., vitamin E TPGS NF (Eastman). Materials with physicochemical properties similar to those of such TPGS materials may be used as bioavailability enhancers according to the invention. Blends of such materials may be used as the bioavailability enhancer component of these extrudates.

The solid dispersion extrudates of the invention may be made as needed to satisfy particular pharmaceutical needs, adjusting the relative amounts of the active compound, polymer carrier, solubilizer, and bioavailability enhancer.

For example, in the solid dispersion extrudates useful in the pharmaceutical compositions of the invention the active compound may be provided in amounts of about 1% w/w to about 50%, about 2% to about 40%, about 5% to about 35%, about 10% to about 30%, about 10% to about 20%, about 10% to about 15%, or about 15% to about 25% w/w. In particular embodiments, the active compound can be about 5%, about 7.5%, about 10%, about 12.5%, about 15%, about 20%, about 25%, or about 30% w/w in the solid dispersion extrudate. The amount of the active compound should not substantially degrade the physically properties of the overall extrudate feed material for the hot melt process.

In the solid dispersion extrudates useful in the pharmaceutical compositions of the invention the polymer carrier may be provided in amounts of about 40% to about 80%, about 45% to about 75%, about 50% to about 70%, about 60% to about 80%, or about 40% to about 60% w/w. In particular embodiments, the polymer carrier can be about 40%, about 45%, about 50%, about 55%, about 60%, about 65%, or about 70% w/w in the solid dispersion extrudate.

In the solid dispersion extrudates useful in the pharmaceutical compositions of the invention the solubilizer may be provided in amounts of about 1% to about 20%, about 5% to about 15%, about 7.5% to about 12.5%, or about 10% to about 20% w/w. In particular embodiments, the solubilizer can be about 5%, about 7.5%, about 10%, about 12.5%, about 15%, or about 20% w/w in the solid dispersion extrudate.

In the solid dispersion extrudates useful in the pharmaceutical compositions of the invention the bioavailability enhancer may be provided in amounts of about 1% to about 20%, about 5% to about 15%, about 7.5% to about 12.5%, or about 10% to about 20% w/w. In particular embodiments, the solubilizer can be about 5%, about 7.5%, about 10%, about 12.5%, about 15%, or about 20% w/w in the solid dispersion extrudate.

Thus, in one aspect the invention provides a solid dispersion extrudate, comprising an active compound selected from:

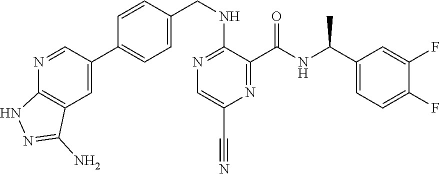 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide,

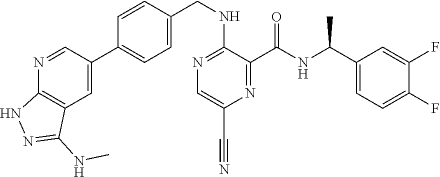 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and

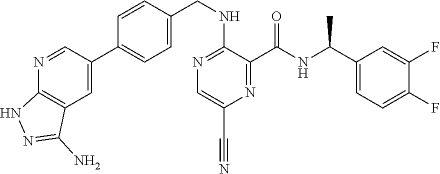 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
   and pharmaceutically acceptable salts of any of the foregoing;

the extrudate further comprising copovidone, PEG 1500, and d-α-tocopheryl polyethylene glycol 1000 succinate.

In various embodiments, the solid dispersion extrudate can comprise:

(a) about 5% to about 15% w/w of the active compound;

(b) about 10% to about 20% of PEG 1500;

(c) about 60% to about 80% w/w of copovidone; and

(d) about 5% to about 15% w/w of d-α-tocopheryl polyethylene glycol 1000 succinate.

In some embodiments, the solid dispersion extrudate comprises about 10% of the active compound.

In some embodiments, the solid dispersion extrudate comprises about 10% of PEG 1500.

In some embodiments, the solid dispersion extrudate comprises about 70% of copovidone.

In some embodiments, the solid dispersion extrudate comprises about 10% of d-α-tocopheryl polyethylene glycol 1000 succinate.

In some embodiments, the pharmaceutical composition comprises a solid dispersion extrudate that is substantially amorphous. In some embodiments, the pharmaceutical composition comprises a solid dispersion extrudate that is amorphous. Determination of the the solid state of the solid dispersion extrudate, such as determination whether the extrudate is substantially amorphous, or amorphous, may be assessed using methods available in the art, such as x-ray powder diffraction (XRPD) analysis or optical microscopy.

In some embodiments, the pharmaceutical composition comprises a solid dispersion extrudate that contains no microcrystalline domains of the compound. In some embodiments, the pharmaceutical composition comprises a solid dispersion extrudate that contains microcrystalline domains of the compound. Existence of crystalline domains in the extrudate may be assessed using methods available in the art, such as Raman spectroscopy analysis and micro-Raman spectroscopy analysis.

### Process of Preparation of Solid Dispersions

The hot melt process is a process of increasing importance in the pharmaceutical industry, as it can enable the use, as active pharmaceutical ingredients (API), of compounds that have physicochemical properties that might otherwise limit their use in pharmaceutical applications. Prime among these properties is solubility, where low solubility of a compound can profoundly limit its bioavailability in a patient, impairing its therapeutic utility and perhaps even precluding use of the compound in the clinic. Thus, overcoming solubility limitations is an important focus of pharmaceutical development.

One method that has been used to improve the bioavailability of some compounds is the hot melt extrusion method. A general overview of this method is given, for example, in K. Kolter, M. Karl. and A. Gryczke, Hot-Melt Extrusion with BASF Pharma Polymers, Extrusion Compendium, 2^{nd} Revised and Enlarged Edition, BASF SE, October 2012 (ISBN 978-3-00-039415-7). See also, e.g., Crowley et al., Drug Devel and Industrial Pharmacy, 2007, 33:909-926, Lang et al., Drug Devel and Industrial Pharmacy, 2014, 40(9):1122-1155, and Madan et al., Asian J. Pharm. Sci., 2012, 7(2):123-133. In general, the process involves providing feedstocks of a polymeric material (usually amorphous) and the compound of interest (often crystalline) to a device that can mix and apply shear stress to the two feed materials to provide a mixture, heating the mixed material, and pressing or extruding the mixed materials, to yield a substantially amorphous dispersion of the compound of interest in the polymer carrier.

Preparation of the solid dispersion extrudates of the invention can be conducted at temperatures suitable for the materials constituting the composition of the dispersion, particularly to avoid undesirable physical degradation of the active compound or of the components of the composition. The extruding is carried out in an extruder operating with a melt temperature ranging about 95° C. to about 160° C. For example, the hot melt process may be conducted at melt temperatures in the range of about 130° C. to about 160° C., about 140° C. to about 150° C., about 140° C., about 145° C., or about 150° C. For example, the the extruding may be carried out in an extruder operating with a barrel temperature comprising stages ranging about 35° C. to about 160° C.

Suitable hot melt procedures may be used on hot melt extrusion apparatus commercially available to the skilled person. The active compound may be combined with the polymer carrier, solubilizer, and bioavailability enhancer, and fed together to the extrusion apparatus. Or the active compound may be provided as a separate feed from the polymeric materials. The feed materials may be extruded using a co-rotating twin screw extruder. Recirculation time may be about 5 minutes to about 15 minutes, for example about 10 minutes. The extrudate may then be chopped or milled into fine particles or pellets upon extrusion.

### Formulations

The present invention also provides pharmaceutical compositions comprising one or more provided compounds in the form of a solid dispersion extrudate, and one or more pharmaceutically acceptable carriers or excipients.

Active compounds can thus be prepared and administered in a wide variety of enteral (e.g., oral, rectal), parenteral, and topical dosage forms. In some embodiments, the provided compositions are administered orally. In some embodiments, the compositions described herein are administered by inhalation, for example, intranasally. In some embodiments, the compositions are administered transdermally. It is also envisioned that multiple routes of administration can be used to administer the compounds using compositions of the invention.

In general, the solid dispersion extrudate in the pharmaceutical compositions, will be provided as a powder or particulate material of preferred dimension and handling characteristics, depending on the intended composition type and the method of administration of the composition. Thus, in one embodiment the solid dispersion extrudate is subjected to a step of comminution or milling by conventional means, which produces a comminuted or milled extrudate material, with the appearance of a powder, particulate, or pellet material. This powder, particulate, or pellet material can then be formulated into pharmaceutical compositions with one or more pharmaceutically acceptable excipients as described herein.

For preparing pharmaceutical compositions using the solid dispersion extrudates described herein, pharmaceutically acceptable excipients can be either solid or liquid. Solid form preparations include powders, tablets, pills, capsules, cachets, suppositories, and dispersible granules. In some embodiments, a solid carrier is one or more substances, which may also act as diluents, flavoring agents, binders, preservatives, tablet disintegrating agents, or an encapsulating material.

In some embodiments, when the composition is a powder or particulate, the carrier is a finely divided solid in a mixture with the finely divided active component. In some embodiments, when the composition is formulated for a tablet, the active component is mixed with the carrier having the necessary binding properties in suitable proportions and compacted in the shape and size desired.

In some embodiments, tablets, powders, capsules, pills, cachets, and/or lozenges are used as solid dosage forms suitable for oral administration. In some embodiments, provided powders, capsules, and tablets contain from 5% to 70% of the active compound. Suitable pharmaceutically acceptable excipients may be selected from known GRAS ingredients, including, for example, magnesium carbonate, magnesium stearate, talc, sugars (such as glucose, lactose), pectins, dextrins, starches, gelatins, gums (such as tragacanth), celluloses (such as methylcellulose, sodium carboxymethylcellulose), low melting waxes, cocoa butter, and the like. Microcrystalline cellulose, for example, can be used to improve powder flow characteristics for capsule filling or tablet compression.

In some embodiments, the pharmaceutical composition is in the form of a capsule. Pharmaceutically acceptable capsules of various types are known in the art. For example, capsules of hydroxypropyl methylcellulose or gelatin may be used. Capsules are desirably suitable for oral administration to patients. For example, capsules may be size 00 or size 1.

Orally administrable forms of the product can be formulated as needed to provide a desired amount of the active moiety of the included active compound. For example, such forms as capsules or tablets may be formulated to contain about 1 mg to about 1,000 mg, about 5 mg to about 500 mg, about 5 mg to about 400 mg, or about 5 mg to about 200 mg of the active moiety. In some embodiments, such forms as capsules or tablets may be formulated to deliver about 1 mg to about 1,000 mg, about 5 mg to about 500 mg, about 5 mg to about 400 mg, or about 5 mg to about 200 mg of the active moiety when administered to a patient. In some embodiments, the orally administrable form may provide a dose of about 5 mg, about 10 mg, about 20 mg, about 25 mg, about 40 mg, about 50 mg, about 60 mg, about 70 mg, about 75 mg, about 80 mg, about 100 mg, about 125 mg, about 150 mg, about 200 mg, about 250 mg, about 300 mg, about 400 mg, about 500 mg, or about 1,000 mg of the active moiety.

Pharmaceutical admixtures suitable for use in the present invention include those described, for example, in Pharmaceutical Sciences (17^{th} Ed., Mack Pub. Co., Easton, Pa.) and WO 96/05309, each of which is hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations intended for conversion shortly before use to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In some embodiments, provided pharmaceutical compositions are in unit dosage form. In such form the composition is subdivided into unit doses containing appropriate quantities of the active component. The unit dosage form can be a packaged preparation, the package containing discrete quantities of a pharmaceutical composition, such as packeted tablets, capsules, and powders. In some embodiments, the unit dosage form is a capsule, tablet, cachet, or lozenge itself, or it is the appropriate number of any of these in packaged form.

The quantity of active compound in a unit dosage form may be varied or adjusted from 0.1 mg to 10,000 mg, more typically 1.0 mg to 2,000 mg, most typically 10 mg to 1,000 mg, according to the particular application and the potency of the active component. The unit dosage may be about 5 mg to about 500 mg, about 5 mg to about 400 mg, or about 5 mg to about 200 mg of the active compound. In some embodiments, provided compositions contain other compatible therapeutic agents at doses calculated to be effective for a given purpose.

### Effective Dosages

Pharmaceutical compositions according to the invention include compositions in which the active compound is provided in a therapeutically effective amount, or in an amount effective to achieve its intended purpose. The actual amount effective for a particular application will depend, inter alia, on the condition being treated. In certain embodiments, when administered in methods to treat cancer, the compositions will contain an amount of active compound effective to achieve the desired result (e.g. decreasing the number of cancer cells in a subject).

The dosage and frequency (single or multiple doses) of administered to a mammal can vary depending upon a variety of factors, including a disease that results in increased activity of PDK1-PIF-mediated substrate interaction-dependent cell survival pathways, whether the mammal suffers from another disease, and its route of administration; size, age, sex, health, body weight, body mass index, and diet of the recipient; nature and extent of symptoms of the disease being treated (e.g., cancer), kind of concurrent treatment, complications from the disease being treated or other health-related problems. Other therapeutic regimens or agents can be used in conjunction with the methods and compounds of the invention.

For any compound useful in the compositions described herein, a therapeutically effective amount of the compound may be initially assessed using cell culture assays. Target concentrations will be those concentrations of active compound(s) that can reduce the activity of PDK1-PIF-mediated substrate interaction-dependent cell survival pathways, as measured, for example, using the methods described in the art.

Therapeutically effective amounts for use in humans may be determined from animal models. For example, a dose for humans can be formulated to achieve a concentration that has been found to be effective in animals. The dosage in humans can be adjusted by monitoring PDK1 inhibition and adjusting the dosage upwards or downwards, as described above.

Dosages may be varied depending upon the requirements of the patient and the compound being employed. In some embodiments, the dose administered to a patient is sufficient to effect a beneficial therapeutic response in the patient over time. The size of the dose also will be determined by the existence, nature, and extent of any adverse side-effects. In some embodiments, treatment is initiated with smaller dosages that are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

Thus, in one embodiment, an effective amount of a pharmaceutical composition as described herein may be administered to the patient according to an intermittent dosing regimen, in which the dosing regimen comprises administering the composition once or twice weekly and the amount of the composition administered each week is about 1 mg to about 1,000 mg. In another embodiment, an effective amount of a pharmaceutical composition as described herein may be administered to the patient according to an intermittent dosing regimen, in which the dosing regimen comprises administering the composition once or twice weekly and the amount of the composition administered each week is about 5 mg to about 500 mg, about 5 mg to about 400 mg, or about 5 mg to about 200 mg. In some embodiments, the pharmaceutical composition may be administered in doses of about 5 mg, about 10 mg, about 20 mg, about 25 mg, about 40 mg, about 50 mg, about 60 mg, about 70 mg, about 75 mg, about 80 mg, about 100 mg, about 125 mg, about 150 mg, about 200 mg, about 250 mg, about 300 mg, about 400 mg, or about 500 mg, or a range between any of the preceding values, for example, between about 10 mg and about 40 mg, between about 100 mg and about 125 mg, between about 50 mg and about 400 mg, or the like.

### Combinations

Formulations of active compounds as described herein can be administered alone, or can be coadministered to a patient along with one or more other pharmaceutically active agents. Coadministration is meant to include simultaneous or sequential administration of such compounds individually or in combination (more than one compound).

Thus, in another aspect, the invention provides methods comprising administering an active compound as described herein or pharmaceutical compositions provided herein in combination with one or more second active agents, and/or in combination with radiation therapy or surgery.

In another aspect, the invention provides a pharmaceutical composition for use in a combinational therapy of treating cancer in a subject, comprising a formulation including a solid dispersion extrudate comprising an active compound as described herein and a pharmaceutically acceptable carrier, in which the combinational therapy further comprises an effective amount of a second anti-cancer agent.

The invention also encompasses therapies in which a patient may be administered an effective amount of a combination of a formulation comprising an active compound as described herein and a second anti-cancer agent. In such combinational therapy, it is possible to administer amounts of each of the agents in the combination that are sub-therapeutic if such agents were to be administered alone, but that in combination the agents act in an additive or supra-additive manner to be therapeutically effective. However, some combinations may employ compounds in amounts that would otherwise be considered therapeutically effective by themselves, yet the combination proves to be more efficacious. In cancers, particularly, a standard of care may be altered by combination of agents, such that a treatment that is effective in some subset of patients becomes transformed into a new standard of care that is effective in a larger set of patients such as by prolonging life or by achieving a higher probability of remission.

Effective combinations of active compounds as described herein with other agents may be identified through preclinical and clinical testing of the combinations, and will depend on many factors, including disease type and stage of development, overall health of the patient, toxicities and side effects of the agents, and the like.

Examples of chemotherapeutic anticancer agents that may be used as second active agents in combination with the active compounds described herein include, but are not limited to, alkylating agents (e.g., mechlorethamine, chlorambucil, cyclophosphamide, melphalan, ifosfamide), antimetabolites (e.g., methotrexate), aurora kinase inhibitors (e.g., ZM447439, hesperidin, VX-680 AZD1152); purine antagonists and pyrimidine antagonists (e.g., 6-mercaptopurine, 5-fluorouracil (5-FU), cytarabine (Ara-C), gemcitabine), spindle poisons (e.g., vinblastine, vincristine, vinorelbine, paclitaxel), podophyllotoxins (e.g., etoposide, irinotecan, topotecan), antibiotics (e.g., doxorubicin, daunorubicin, bleomycin, mitomycin), nitrosoureas (e.g., carmustine, lomustine), inorganic ions (e.g., platinum complexes such as cisplatin, carboplatin), enzymes (e.g., asparaginase), hormones (e.g., tamoxifen, leuprolide, flutamide, and megestrol), topoisomerase II inhibitors or poisons, EGFR (Her1, ErbB-1) inhibitors (e.g., gefitinib), antibodies (e.g., bevacizumab, rituximab), IMIDs (e.g., thalidomide, lenalidomide), various targeted agents (e.g., HDAC inhibitors such as vorinostat), Bcl-2 inhibitors, VEGF inhibitors, proteasome inhibitors (e.g., bortezomib), cyclin-dependent kinase (cdk) inhibitors (e.g., seliciclib), quinolone derivatives (e.g., vosaroxin), and dexamethasone.

In other embodiments, active compounds as described herein may be used in combination therapy with PDK1 inhibitors, e.g., GSK2334470 (GlaxoSmithKline), BX-795, BX-912, and BX-320 (Berlex); Akt inhibitors, e.g., MK-2206 (Merck); PI3K inhibitors, e.g., GDC-0941 (pictilisib, Genentech), idelalisib (Zydelig™; Gilead); BTK inhibitors, e.g., GS-4059 (Gilead).

In the treatment of hematological and solid tumors, second agents can include inhibitors of PD-1/PD-L1, for example, nivolumab (Opdivo™), pembrolizumab (Keytruda™, MK-3475), pidilizumab (CT-011), BMS 936559, and MPDL328OA; CTLA-4 (CD152) inhibitors, for example, ipilimumab (Yervoy™) and tremelimumab; and phosphatidylserine inhibitors, for example, bavituximab (PGN401).

In the treatment of acute myelogenous leukemia, second agents include, for example, cytarabine (ara-C), daunorubicin, and vosaroxin.

In the treatment of CLL, second agents include, for example, PCI-32765 (ibrutinib, Imbruvica™).

In the treatment of myelomas, second agents include, for example, lenalidomide (Revlimid™) and bortezomib (Velcade™).

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples that follow and the references to the scientific and patent literature cited herein. It should further be appreciated that the contents of those cited references are incorporated herein by reference to help illustrate the state of the art.

It will be appreciated that for compound preparations described herein, when reverse phase HPLC is used to purify a compound, a compound may exist as a mono-, di-, or tri-trifluoroacetic acid salt.

It will further be appreciated that the present invention contemplates individual compounds described herein. Where individual compounds exemplified are isolated and/or characterized as a salt, for example, as a trifluoroacetic acid salt, the present invention contemplates a free base of the salt, as well as other pharmaceutically acceptable salts of the free base.

The following examples contain important additional information, exemplification and guidance that can be adapted to the practice of this invention in its various embodiments and the equivalents thereof.

### EXAMPLES

Without wishing to be bound by any particular theory, it is believed that active compounds as described herein bind the inactive conformation of PDK1 (IC₅₀<20 nM). The compounds bind deep in the adaptive (allosteric) pocket, causing a distortion in the N-terminal domain thereby perturbing the PIF-pocket and thus negatively modulating PI-independent substrate binding. Compound 2, for example, has been evaluated in a panel of more than 20 cell lines derived from hematologic cancers including acute myelogenous leukemia, multiple myeloma, DLBCL, and Mantle cell lymphoma, and shows strong anti-proliferative activity with EC₅₀, =3-900 nM. Anti-proliferative activity correlated with pathway modulation assessed by inhibition of phosphorylation of PDK1, RSK2, and AKT. Interestingly, inhibition of PDK1 phosphorylation was time-dependent, showing 2-5-fold more inhibition after 24 hours than 4 hours. In addition, Compound 2 produced substantial apoptosis after 24 hours. Compound 2 was compared to the PDK1 inhibitor GSK2334470, showing comparable biochemical potency, but Compound 2 was 10- to 30-fold more potent at inhibiting PDK1 and RSK2 phosphorylation in all cell lines tested. In addition, Compound 2 was at least 10-fold more potent than GSK2334470 in 72-hour viability assays.

In mice, Compound 1 and Compound 2 are orally bioavailable (% F>40%) with a Tₘₐₓ of 4-8 hours and long half-life. Pathway modulation was assessed in vivo using MV4-11 xenografts in mice. Potent pathway modulation was observed at 4 hours and 24 hours after a single oral dose of Compound 1 and Compound 2. Efficacy was assessed by 21-day dosing in MV4-11 xenografts. Both Compound 1 and Compound 2 show dose-related efficacy with TGI reaching 96-97% and partial regression in 70-100% of animals at the highest dose.

Without wishing to be bound by any particular theory, it is believed that targeting the inactive conformation of PDK1 and inhibiting PI-independent substrate binding has broad potential for the treatment of solid and hematologic cancers, especially in contexts in which PDK1 kinase inhibitors or Akt inhibitors are insufficiently effective.

### Example 1

To address the challenges posed by new drug candidates showing unfavorable biopharmaceutical characteristics such as poor water solubility and/or low permeability (C. A. Lipinski et al., Adv. Drug Deliv. Rev., 2012, 64:4-17), the development of advanced drug delivery systems must be taken into consideration not only for clinical trials but also much earlier in preclinical studies. (J. Maas et al., Eur. J. Pharm. Biopharm., 2007, 66:1-10)

While the application of concepts of Biopharmaceutical Classification System (BCS) (G. L. Amidon et al., Pharm. Res., 1995, 12:413-420) has made the development of molecules displaying solubility-limited (BCS class II) or permeability-limited (BCS class III) oral bioavailability almost a common practice, that of poorly soluble and low permeable compounds (BCS class IV) still remains problematic. In fact, to develop effective drug products suitable for both preclinical and clinical studies, specific delivery systems combining strategies to improve the bioavailability of BCS class II as well as BCS class III compounds must be designed.

This study describes development and scale-up of an amorphous solid dispersion of a poorly soluble and low permeable active compound (Compound 1), with the goal of enabling conduct of toxicology studies in dog and formulation development for first time in human (FTIH) testing.

Previous investigations showed that approaches used to improve the absorption of the BCS class IV compounds such as traditional powder suspension, salt screening, drug solubilization, particle size reduction (nano-milling) were not successful in providing sufficient oral bioavailability of Compound 1. In contrast, good results were obtained in rat and dog pharmacokinetic studies using lipid based solutions or hot melt extrusion (HME) formulations including d-α-tocopheryl polyethylene glycol 1000 succinate (TPGS) as a bioavailability enhancer.

TPGS was also used as a plasticizer to improve the physical characteristics of extrudates and to keep the extrusion temperature as low as possible avoiding drug degradation due to excessive exposure to heat.

The optimization of HME formulation composition and process and the scale up were performed to achieve the following objectives:
Assessment of drug load and physicochemical stabilization of extrudate;
Preparation of HME formulation at larger scale to support enabling Phase 1 general toxicology studies in dog;
Providing HME formulation composition and process suitable to be used for FTIH.

The optimization of solid dispersion previously tested in pharmacokinetic studies including 10% of Compound 1, 60% Copovidone, 10% PEG 1500 and 10% TPGS was carried out by extruding different formulation compositions at lab scale using the Thermo Scientific MiniLab II Micro Compounder. The following variables were investigated:
Drug concentration: within the range 10.0% w/w-15.0% w/w;
Concentration of PEG 1500 and TPGS: either 10.0% w/w or 20.0% w/w; and
Extrusion Temperature: 95° C.-140° C.

This initial part of the study allowed selecting two formulations and related processes that were scaled up using the Thermo Fisher Pharma 16 Extruder. Process parameters were:
Temperature of extruder stages: within 35° C.-160° C.;
Screw speed: 700 rpm; and
Feed rate: 9 g/min.

Extruded materials were analyzed for assay and impurities using specifically developed HPLC method and for solid state characterization by Optical Microscopy (OM) and XRPD. In addition, Micro-Raman Spectroscopy was used to confirm the presence of drug micro-crystalline areas in the extrudates.

Finally, the optimized HME formulation along with matching placebo was used for enabling Phase 1 general toxicology studies in dog and for stressed stability and excipient compatibility studies.

Additional studies on previously developed HME solid dispersion of Compound 1 were conducted at small scale to select the most suitable levels of drug and additives (PEG 1500 and TPGS) acting as solubilizers, plasticizers and bioavailability enhancers. Moreover, different extrusion conditions were assessed, and combined effects of temperature and formulation composition investigated (data not shown).

This way, two formulations: Trial 1 and 3, respectively, at 15.0% w/w and 10.0% w/w drug loading were identified and successfully scaled up to 1 Kg along with one at 12.5% w/w drug loading (Trial 2) and one placebo (Trial 4).

Table 1 reports the employed compositions and extrusion parameters and the results of physical and analytical tests performed on the extrudates. Amounts shown are % w/w of the total composition.

TABLE 1

| | | | | Trial 4 |
|---|---|---|---|---|
| | Trial 1 | Trial 2 | Trial 3 | (Placebo) |
| Drug (% w/w) | 15.0 | 12.5 | 10.0 | — |
| Copovidone (% w/w) | 55.0 | 57.5 | 60.0 | 70.0 |
| TPGS (% w/w) | 20.0 | 20.0 | 20.0 | 20.0 |
| PEG 1500 (% w/w) | 10.0 | 10.0 | 10.0 | 10.0 |
| Rod Appearance OM | Dark yellow rod | Dark yellow rod | Dark yellow | Translucent |
| | with black dots | with black dots | homogeneous | clear |
| | | | rod | homogeneous |
| | | | | rod |
| XRPD | Amorphous | Amorphous | Amorphous | — |
| | Dispersion | Dispersion | Dispersion | |
| Raman Spectroscopy | Presence of | Presence of | No Micro- | — |
| | Micro- | Micro- | crystalline areas | |
| | crystalline areas | crystalline areas | detected | |
| Assay at T = 0 | — | — | 9.12 | — |
| (% w/w) | | | | |
| Total Impurities at | — | — | 0.73 | — |
| T = 0 (% w/w) | | | | |
| Assay at T = 1 Month | — | — | 9.41 | — |
| (% w/w) | | | | |
| Total Impurities at | — | — | 0.82 | — |
| T = 1 Month (% w/w) | | | | |

Although XRPD analysis showed an amorphous pattern, and no peaks related to crystalline drug substance were detected, the optical microscopy observation revealed the presence of black spots in the extrudates of Trials 1 (15.0% drug load) and Trial 2 (12.5% drug load). Micro-Raman spectroscopy analysis confirmed these to be micro-crystalline areas or domains of Compound 1. In contrast, such micro-crystalline domains were not detected for Trial 3 (10.0% drug load).

Hence, the Trial 3 and Placebo Trial 4 extrudates were suitably milled and employed for Phase 1-enabling toxicology studies in dog. The same batches were assessed for stability at refrigerated conditions (5° C./Ambient RH) and showed neither changes in appearance nor significant increase of total impurities along the duration of toxicology study (Table 1).

Finally, Trial 3 extrudate was also used for a prototype stressed stability study as a first step of clinical formulation development. Three excipient compositions (1-3) were employed to make capsules at 1 mg and 50 mg dose strengths (Table 2), which were packaged into HDPE bottles and screened for chemical compatibility. No significant increase of total impurities was detected after storage for 1 month at 40° C. and 75% RH for all compositions.

TABLE 2

| | Composition 1 | | Composition 2 | | Composition 3 | |
|---|---|---|---|---|---|---|
| Dose Strength | Low | High | Low | High | Low | High |
| Extrudate | 10.98 | 549.45 | 10.98 | 549.45 | 10.98 | 549.45 |
| Trial 9 (mg) | | | | | | |
| (9.16% drug | | | | | | |
| loading) | | | | | | |
| Microcrystalline | — | — | — | — | 80.12 | 29.10 |
| Cellulose (mg) | | | | | | |
| Pregelatinized | — | — | 80.12 | 29.10 | — | — |
| Starch (mg) | | | | | | |
| Croscarmellose | 80.12 | 29.10 | — | — | — | — |
| (mg) | | | | | | |
| Magnesium | 8.90 | 2.91 | 8.90 | 2.91 | — | — |
| Stearate (mg) | | | | | | |
| HPMC Capsule | 1 EA | 1 EA | 1 EA | 1 EA | 1 EA | 1 EA |
| shell opaque | | | | | | |
| white size 00 | | | | | | |
| Total Impurities | 2.1 | 1.8 | 2.0 | 1.9 | 2.2 | 1.9 |
| at T = 0 (%) | | | | | | |
| Total Impurities | 2.3 | 1.8 | 2.1 | 1.8 | 2.4 | 1.8 |
| at T = 1 Month | | | | | | |
| (%) (Storage | | | | | | |
| 40° C./75% RH) | | | | | | |

An HME formulation of a BCS class IV compound, Compound 1, was successfully optimized and scaled up.

The amorphous solid dispersions including 10% w/w of drug, copovidone as a polymer carrier, PEG 1500 as a solubilizer/plasticizer, and TPGS as a bioavailability enhancer proved suitable in terms of scalability, analytical results, and stability.

The application of this bioavailability-enhancing formulation approach allowed manufacture of a test drug product at suitable scale to support toxicology studies in dog as well as to conduct preliminary formulation investigations for FTIH testing of Compound 1 despite its otherwise poor developability properties.

### Example 2

BCS class IV drugs exhibit many characteristics that are problematic for effective oral delivery which most likely leads to low and variable bioavailability. Appropriate formulation design is, therefore, of key importance to progress an investigational product from pre-clinical phase into the clinic for testing in human patients.

Application of different technologies was investigated seeking to improve the oral bioavailability of a BCS class IV active compound (Compound 1) which exhibits very low solubility properties (<0.1 μg/mL over the physiological pH range) and low permeability. To overcome the inherent hurdles posed by this class of drugs, three alternative formulation approaches were screened: lipid-based formulation, nano-suspension, and solid dispersion via hot melt extrusion (HME).

In addition, Vitamin E TPGS has been employed based on its solubility, absorption, and permeation enhancement properties. Pre-clinical pharmacokinetics (PK) investigations were used as a tool for screening the formulations by assessing in vivo their performances in terms of systemic exposure and to allow more flexibility of dose range. Two PK studies were performed: one in rat following single oral administration of compound A as a liquid formulation, and a second in dog following single oral administration of compound A as a solid dosage form.

Lipid based formulations were developed through a solubility screening study investigating excipients of different nature such as lipidic, solvents, surfactants (anionic, non-ionic, and polymeric) and Vit E TPGS. Solubility was assessed in saturated solution by HPLC. The most promising vehicles were then mixed at different ratios to achieve the highest possible concentration of API in solution.

Different nano-suspension compositions were prepared by wet bead milling (WBM) in water and characterized for Particle Size Distribution (PSD) as shown in Table 3, and assay and Impurities profile at initial time point and after storing for seven days at 5° C.

TABLE 3

| Prototype | 1 | 2 | 3 | 4 |
|---|---|---|---|---|
| Compositions | | | | |
| Compound 1 | 20% | 20% | 20% | 10% |
| PVP | 1% | — | 1% | 1% |
| HPMC | — | 1% | — | — |
| DOSS | 0.25 | 0.25% | — | 0.25% |
| POLOXAMER | — | — | 0.25% | — |
| Particle Size Distribution | | | | |
| D10 (μm) | 0.067 | 0.07 | N/A | 0.063 |
| D50 (μm) | 0.138 | 0.152 | N/A | 0.125 |
| D90 (μm) | 0.297 | 0.437 | N/A | 0.259 |

In screening for solid dispersion preparation, the ability of excipients (different mixtures of suitable carrier and additives as per Table 4) to molecularly disperse Compound 1 was tested via DSC thermo-cycling (heating from 0° C. up to 260° C. and back).

TABLE 4

| Trial | 1 | 2 | 3 | 4 | 5 |
|---|---|---|---|---|---|
| API | 10% | 10% | 10% | 10% | 10% |
| Polymer | Copovidone | Copovidone | PEG 1500 | Copovidone | Copovidone |
| | 70% | 70% | 50% | 70% | 70% |
| Additive | PEG 1500 | TPGS 30% | TPGS 50% | Poloxamer | Poloxamer |
| | 30% | | | 188 30% | 273 30% |
| Trial | 6 | 7 | 8 | 9 | 10 |
| API | 10% | 10% | 10% | 10% | 15-20% |
| Polymer | Copovidone | Copovidone | Copovidone | Copovidone | Copovidone |
| | 70% | 70% | 90% | 90% | 70% |
| Additive | PEG6000 | Decanoic Acid | PEO 10% | HAS 10% | Best additives |
| | 30% | 30% | | | 30% |

The two most promising compositions were then processed via HME on lab scale extruder using different processing conditions.

The extruded materials were characterized for solid state by DSC and XRPD, and for assay and impurities. Both formulations resulted in amorphous solid dispersions that were physically and chemically stable for the duration of study.

### Pharmacokinetics

Three naïve male Crl:CD (SD) rats were dosed orally with Compound 1 formulated as a solution in PEG 400, lipid-based liquid formulation, and as a nano-milling suspension in a crossover study design.

Three non-naïve male beagle dogs were dosed orally with Compound 1 formulated as solid formulations filled into a capsule and as solution in PEG400 in a crossover study design. During each PK study, an individual serial plasma profile was drawn from each animal up to at least 24 hours after dosing.

All plasma samples were assayed for Compound 1 using a qualified method based on protein precipitation, followed by LC/MS-MS analysis, then PK elaboration was performed by non-compartmental analysis using Phoenix® WinNonlin® (Certara L.P.).

Results

The three lead formulation candidates were tested in PK studies in male rats and dogs.

For the rat PK study a liquid oral dosage form was dosed to facilitate administration to the animal.

Among lipid-based liquid formulations, the following composition was selected as the best in terms of achieved concentration and physical stability: PEG400 (85% w/w), TPGS (2.5% w/w), PEG 1500 (2.5% w/w), DMSO (5% w/w), and NMP (5% w/w).

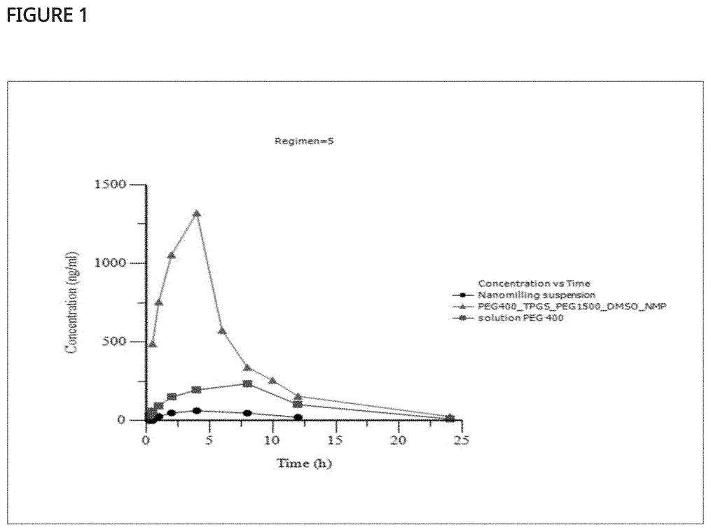

Nanosuspension compositions 1 and 4 (Table 1) resulted in being the most promising in terms of PSD, solid state and chemical analysis, after preparation as well as upon storage. Considering the advantage of higher drug loading, prototype 1 was selected for the study. Solution in PEG was used as a reference formulation. The obtained PK results as concentration versus time are reported in FIG. 1.

At the same oral dose tested, systemic exposure reached with the lipid composition (red profile) was significantly higher than that obtained with the other two liquid formulations: Cₘₐₓ was found to be 21- and 6-fold higher, and AUCₗₐₛₜ 13- and 3-fold higher, with the lipid composition when compared to the values obtained with nanosuspension and solution in PEG400, respectively.

For the PK study in dog, a single oral administration of solid dosage form was considered acceptable. Therefore, two different HME compositions were tested while the solution in PEG 400 remained the reference formulation.

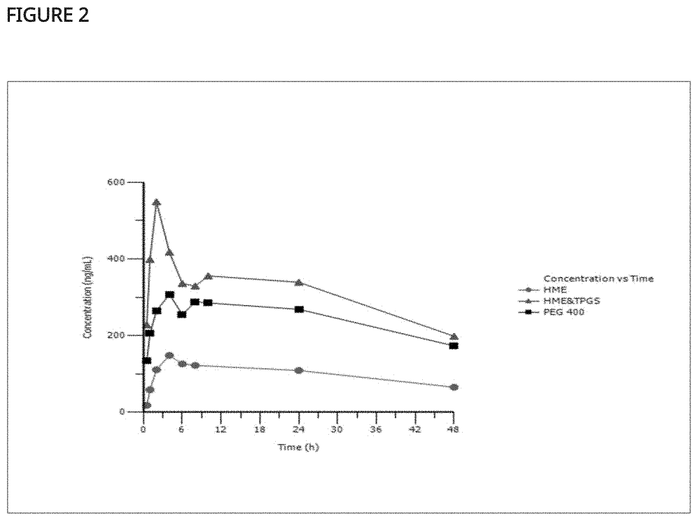

To evaluate the effect of additives to the HME composition, extrudates prepared:
(1) 10% active and 90% of Kollidon® VA64; (see HME, FIG. 2)
(2) 10% active, 60% Kollidon® VA64, 20% PEG 1500, and 10% TPGS; (see HME & TPGS, FIG. 2)
   and tested in vivo. The obtained PK results as concentration vs time are reported in FIG. 2.

For the dog study the amorphous dispersion containing TPGS gave significantly higher exposure versus both the simple binary HME with polymer and the solution in PEG400. Specifically, with HME containing TPGS test item, systemic exposure (either in terms of Cₘₐₓ and AUCₗₐₛₜ) was 2- and 4-fold higher than values obtained with solution in PEG400 and with HME simple composition, respectively.

The addition of TPGS significantly enhanced bioavailability of Compound 1 in rat and dog in both test formulations: lipid-based solution and amorphous solid dispersion.

### Example 3

Sample hot melt extrudates were made using a Thermo Scientific MiniLab II Micro Compounder:

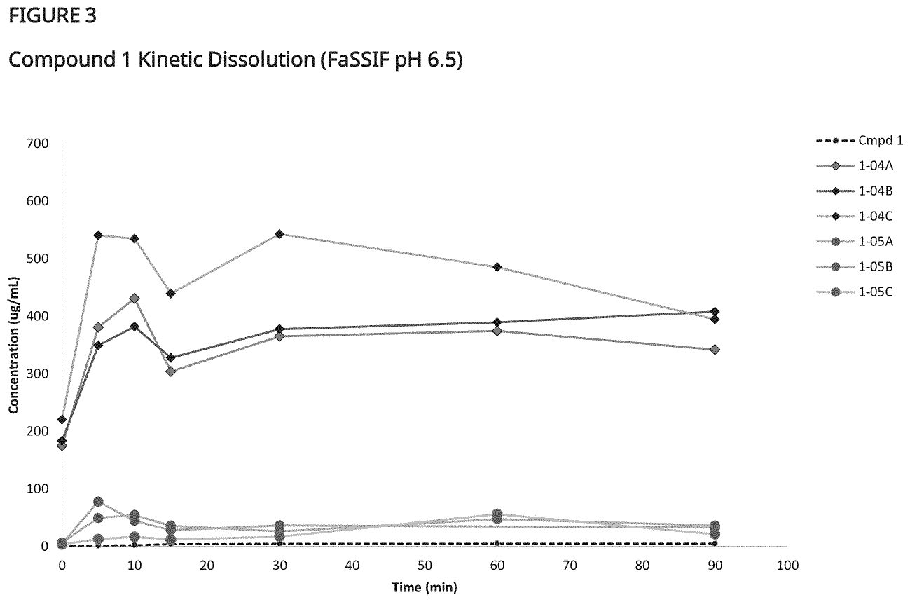

10% Compound 1/10% PEG 1500/10% TPGS/70% VA-64 (1-04A, 1-0413, and 1-04C, see Table 5; FIG. 3)

10% Compound 1/10% TPGS/80% HPMCAS (MF) (1-05A, 1-05B, and 1-05C, see Table 5; FIG. 3)

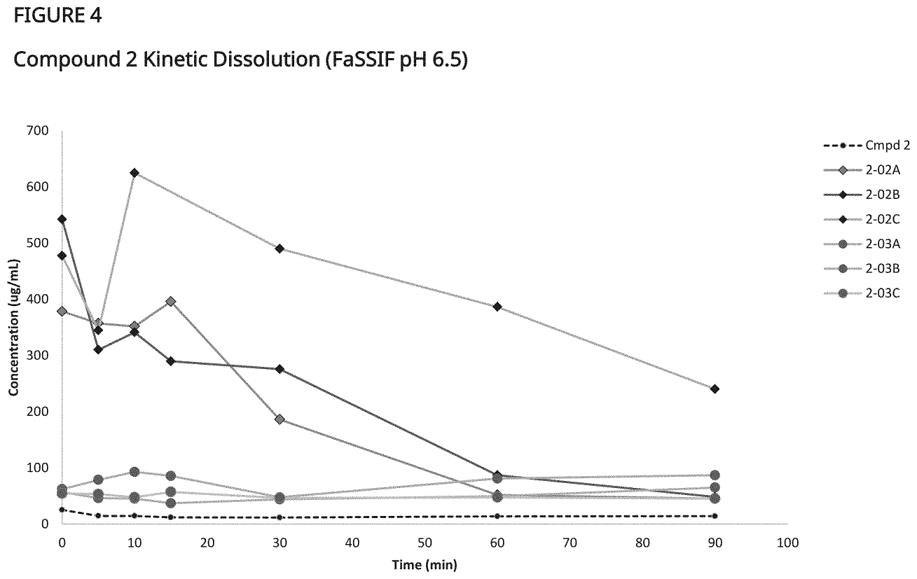

10% Compound 2/10% PEG 1500/10% TPGS/70% VA-64 (2-02A, 2-02B, and 2-02C, see Table 6; FIG. 4)

10% Compound 2/10% TPGS/80% HPMCAS (MF). (2-03A, 2-03B, and 2-03C, see Table 6; FIG. 4)

HPMCAS (MF) is a hydroxypropyl methyl cellulose acetate succinate powder with solubility at pH 6.0 and above. Samples of each formulation were prepared by extrusion at 130° C., 140° C., and 150° C. HME samples were then subjected to kinetic dissolution under the following conditions:

| | |
|---|---|
| Dissolution Media | pH 6.5 FaSSIF |
| Temperature | 37° C. |
| 1 mL FaSSIF to 10 mg | HME |
| Thermo Shaker | 500 rpm |
| Centrifuge | 1 minute at 12,400 rpm |
| Sample Dilution | 1:10 using acetonitrile |
| Sample Times | 0, 5, 10, 15, 30, 60, & 90 minutes |

Fasted State Simulated Intestinal Fluid (FaSSilF; 3 mM sodium taurocholate, 0.75 mM lecithin, at 270±10 mOsmol and pH 6.5) was obtained from SIGMA ALDRICH; Allentown, Pa.

Results are shown in FIGS. 3 and 4. The samples were also assayed and tested for purity using conventional HPLC methods. These data are shown in Table 5 (Compound 1) and Table 6 (Compound 2). It is evident that the HPMCAS samples were sensitive to extrusion temperature, with both assay and purity declining as temperature was increased.

TABLE 5

| | | | Assay | Purity |
|---|---|---|---|---|
| Sample | Carrier | Condition | (%) | (%) |
| 1-04A (FIG. 3) | VA-64 | 130° C. | 91.29 | 96.75 |
| 1-04B (FIG. 3) | VA-64 | 140° C. | 101.73 | 97.91 |
| 1-04C (FIG. 3) | VA-64 | 150° C. | 98.55 | 97.81 |
| 1-05A (FIG. 3) | HPMCAS | 130° C. | 95.91 | 93.85 |
| 1-05B (FIG. 3) | HPMCAS | 140° C. | 85.50 | 87.80 |
| 1-05C (FIG. 3) | HPMCAS | 150° C. | 76.51 | 80.71 |
| Working Standard | 100% Methanol | RT | | 97.91 |

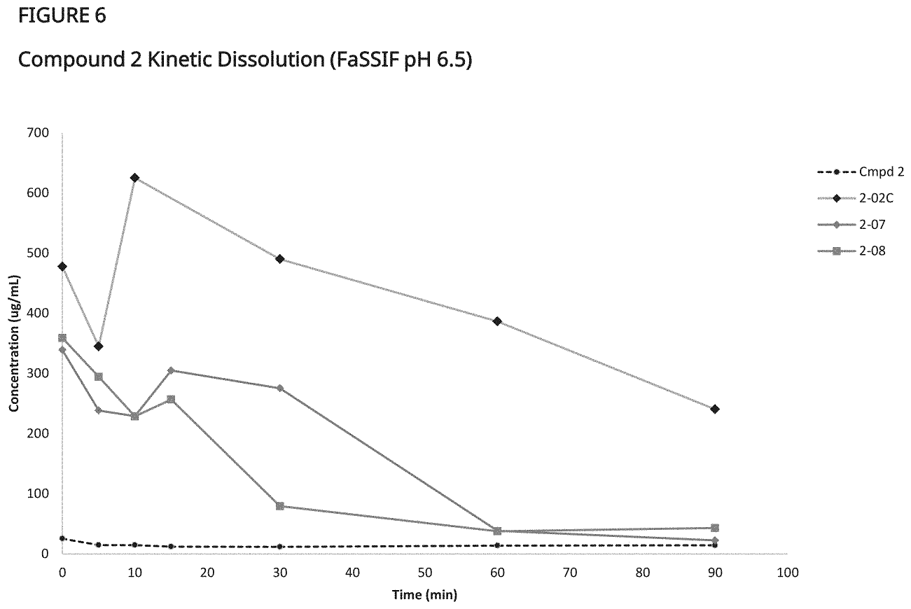

TABLE 6

| | | | Assay | Purity |
|---|---|---|---|---|
| Sample | Carrier | Condition | (%) | (%) |
| 2-02A (FIG. 4) | VA-64 | 130° C. | 89.61 | 97.46 |
| 2-02B (FIG. 4) | VA-64 | 140° C. | 91.80 | 96.88 |
| 2-02C (FIG 4; FIG. 6) | VA-64 | 150° C. | 93.14 | 96.56 |
| 2-03A (FIG. 4) | HPMCAS | 130° C. | 82.51 | 87.28 |
| 2-03B (FIG. 4) | HPMCAS | 140° C. | 73.83 | 80.70 |
| 2-03C (FIG. 4) | HPMCAS | 150° C. | 72.99 | 79.85 |
| Working Standard | 100% Methanol | RT | | 98.86 |

### Example 4

Various concentrations of active compound were tested in TPGS/PEG 1500/VA-64 compositions. Production and kinetic dissolution of the samples were conducted using the methods described in Example 3. As amounts of active compound were increased from 10% w/w to 30% w/w, corresponding reductions were made in the amount of VA-64 from 70% w/w to 50% w/w; amounts of PEG 1500 and TPGS were maintained constant, at 10% w/w each. Extrusion was conducted at 150° C.

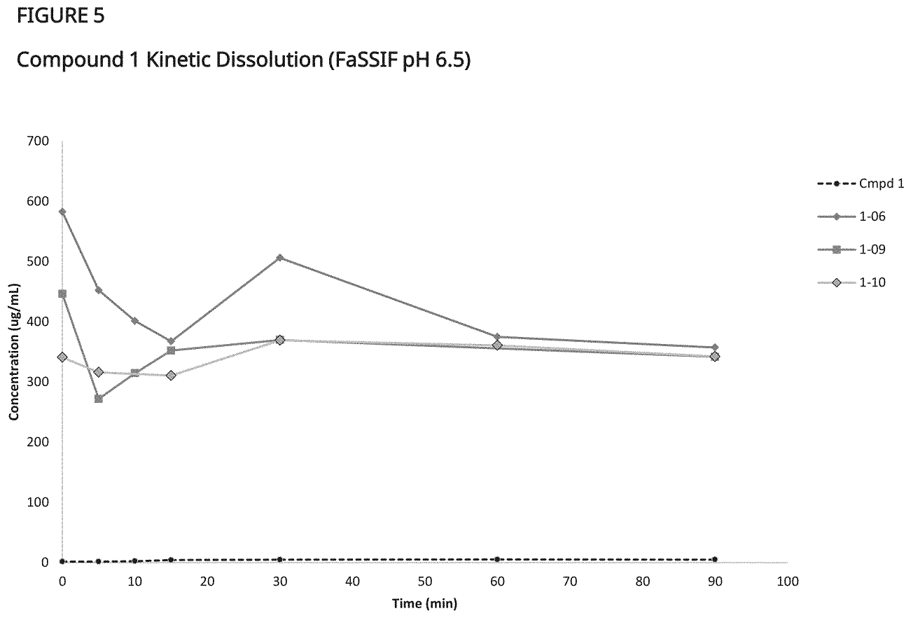

Results are presented in Table 7, with graphical representations of the kinetic dissolution results presented in FIGS. 5 and 6.

TABLE 7

| | | | Assay | Purity |
|---|---|---|---|---|
| Sample | Compound | Condition | (%) | (%) |
| 1-06 (FIG. 5) | 10% Cmpd 1 | 150° C. | 93.5 | 96.91 |
| 1-09 (FIG. 5) | 20% Cmpd 1 | 150° C. | 94.4 | 98.42 |
| 1-10 (FIG. 5) | 30% Cmpd 1 | 150° C. | 95.8 | 98.96 |
| 2-07 (FIG. 6) | 20% Cmpd 2 | 150° C. | 97.7 | 97.67 |
| 2-08 (FIG. 6) | 30% Cmpd 2 | 150° C. | 99.8 | 97.79 |
| Working Standard | Cmpd 1 | RT | N/A | 98.84 |
| Working Standard | Cmpd 2 | RT | N/A | 98.23 |

The data from this experiment demonstrate that various concentrations of active compounds as described herein may be employed in the compositions of the invention as may be needed for particular applications. Dissolution of the compounds from the solid dispersion product was substantially improved relative to dissolution of the compounds from the working standards.

Certain embodiments of the invention are illustrated:

1. A pharmaceutical composition comprising a solid dispersion extrudate comprising an active compound selected from:

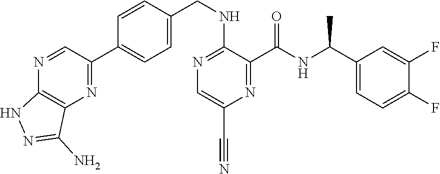 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide,

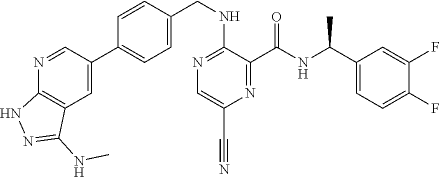 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and

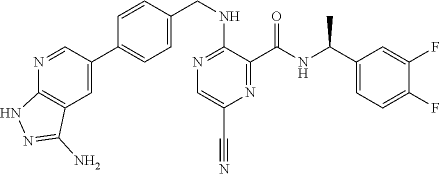 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
   and pharmaceutically acceptable salts of any of the foregoing;
   the extrudate further comprising a polymer carrier, a solubilizer/plasticizer, and a bioavailability enhancer.

2. A pharmaceutical composition as described in embodiment 1, in which the polymer carrier is a vinylpyrrolidinone-vinyl acetate copolymer.

3. A pharmaceutical composition as described in embodiment 2, in which the vinylpyrrolidinone-vinyl acetate copolymer is copovidone.

4. A pharmaceutical composition as described in embodiment 2, in which the amount of vinylpyrrolidinone-vinyl acetate copolymer in the extrudate is about 45% to about 75% w/w.

5. A pharmaceutical composition as described in embodiment 4, in which the amount of vinylpyrrolidinone-vinyl acetate copolymer in the extrudate is about 60% w/w

6. A pharmaceutical composition as described in embodiment 1, in which the solubilizer/plasticizer is PEG 1500.

7. A pharmaceutical composition as described in embodiment 6, in which the amount of PEG 1500 in the extrudate is about 5% to about 25% w/w.

8. A pharmaceutical composition as described in embodiment 7, in which the amount of PEG 1500 in the extrudate is about 20% w/w.

9. A pharmaceutical composition as described in embodiment 1, in which the bioavailability enhancer is d-α-tocopheryl polyethylene glycol 1000 succinate (TPGS).

10. A pharmaceutical composition as described in embodiment 8, in which the amount of TPGS in the extrudate is about 5% to about 25% w/w.

11. A pharmaceutical composition as described in embodiment 9, in which the amount of TPGS in the extrudate is about 10% w/w.

12. A pharmaceutical composition as described in embodiment 1, in which the amount of the active compound in the extrudate is about 5% to about 35% w/w.

13. A pharmaceutical composition as described in embodiment 12, in which the amount of the active compound in the extrudate is about 10% to about 20% w/w.

14. A pharmaceutical composition as described in embodiment 13, in which the amount of the active compound in the extrudate is about 10% w/w.

15. A pharmaceutical composition as described in embodiment 13, in which the amount of the active compound in the extrudate is about 15% w/w.

16. A pharmaceutical composition as described in embodiment 13, in which the amount of the active compound in the extrudate is about 20% w/w.

17. A pharmaceutical composition as described in any of embodiments 1-16, in which the solid dispersion extrudate is substantially amorphous, as determined by x-ray powder diffraction analysis.

18. A pharmaceutical composition as described in any of embodiments 1-116, in which the solid dispersion extrudate contains crystalline domains of the active compound, as determined by Raman spectroscopy analysis.

19. A pharmaceutical composition as described in in any of embodiments 1-16, in which the solid dispersion extrudate contains no crystalline domains of the active compound, as determined by Raman spectroscopy analysis.

20. A pharmaceutical composition as described in embodiment 1, in which the glass transition temperature (Tg) of the solid dispersion extrudate is about 45° C. to about 120° C.

21. A pharmaceutical composition as described in embodiment 1, further comprising one or more pharmaceutically acceptable excipients.

22. A pharmaceutical composition as described in any of embodiments 1-21, comprising (a) about 10% to about 50% w/w of a solid dispersion extrudate comprising an active compound as described herein or a pharmaceutically acceptable salt thereof, a vinylpyrrolidinone-vinyl acetate copolymer, PEG 1500, and d-α-tocopheryl polyethylene glycol 1000 succinate, and (b) about 50% to about 90% w/w of one or more pharmaceutically acceptable excipients.

23. A pharmaceutical composition as described in any of embodiments 1-22, in which the active compound is selected from:

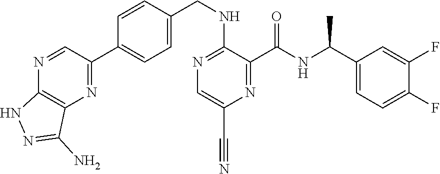 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide, or a pharmaceutically acceptable salt thereof.

24. A pharmaceutical composition as described in any of embodiments 1-22, in which the active compound is selected from:

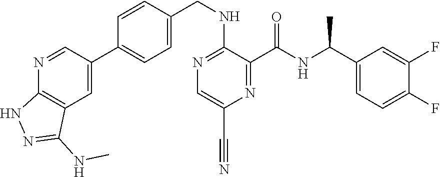 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, or a pharmaceutically acceptable salt thereof.

25. A pharmaceutical composition as described in any of embodiments 1-22, in which the active compound is:

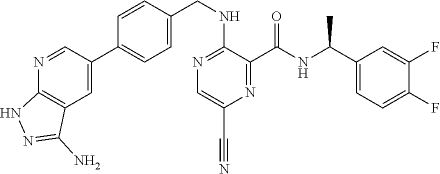 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide, or a pharmaceutically acceptable salt thereof.

26. An orally administrable preparation of an active compound selected from:

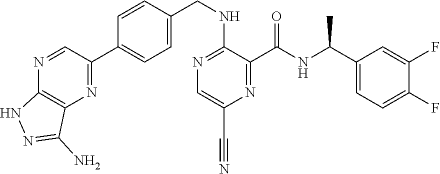 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide,

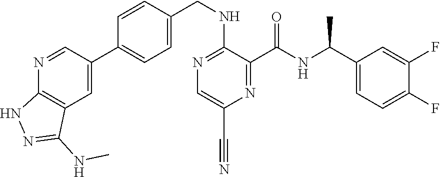 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and

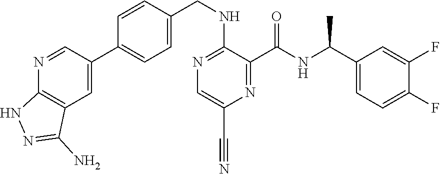 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
   and pharmaceutically acceptable salts of any of the foregoing;

the preparation comprising:
(a) a solid dispersion extrudate comprising the active compound, a polymer carrier, a solubilizer/plasticizer, and a bioavailability enhancer, and
(b) one or more pharmaceutically acceptable excipients.

27. An orally administrable preparation as described in embodiment 26, in which the pharmaceutically acceptable excipients are selected from microcrystalline cellulose, pregelatinized starch, magnesium stearate, and combinations thereof.

28. An orally administrable preparation as described in embodiment 26 or 27, which is a capsule or a tablet.

29. An orally administrable preparation as described in embodiment 28, which is a hydroxypropyl methylcellulose or gelatin capsule.

30. An orally administable preparation as described in any of embodiments 26-29, which comprises about 40% to about of the solid dispersion extrudate and about 60% to about 40% of microcrystalline cellulose.

31. An orally administrable preparation as described in any of embodiments 26-30, providing a dose of about 1 mg to about 500 mg of an active moiety of the active compound.

32. A process for preparing a pharmaceutical composition, which comprises the steps of:

(i) hot melt extruding a mixture of:
a. an active compound selected from:

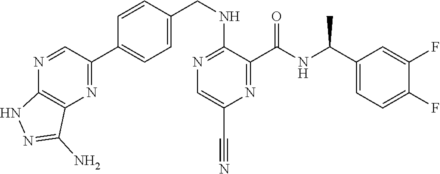 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide,

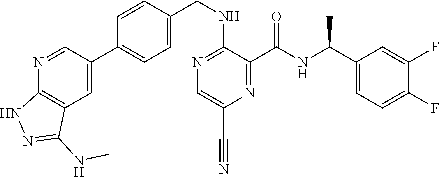 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and

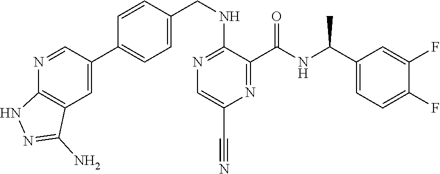 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
   and pharmaceutically acceptable salts of any of the foregoing, and
   b. a polymer carrier, a solubilizer/plasticizer, and a bioavailability enhancer, to form a solid dispersion extrudate; and

(ii) blending the resulting solid dispersion extrudate with one or more pharmaceutically acceptable excipients.

33. A process as described in embodiment 32, in which the polymer carrier is a vinylpyrrolidinone-vinyl acetate copolymer.

34. A process as described in embodiment 32, in which the solubilizer is PEG 1500.

35. A process as described in embodiment 32, in which the bioavailability enhancer is d-α-tocopheryl polyethylene glycol 1000 succinate.

36. A process as described in embodiment 32, in which the extruding is carried out in an extruder operating with a barrel temperature comprising stages ranging about 35° C. to about 160° C.

37. A process as described in embodiment 32, in which the extruding is carried out in an extruder operating with a melt temperature ranging about 95° C. to about 160° C.

38. A method for the treatment of cancer in a patient in need of such treatment, comprising administering an effective amount of a pharmaceutical composition as described in any of embodiments 1-25 or an orally administrable preparation as described in any of embodiments 26-31, to the patient according to an intermittent dosing regimen, in which the dosing regimen comprises administering the composition once or twice weekly and the amount of the composition administered each week is about 1 mg to about 1000 mg.

39. A method as described in embodiment 38, in which the cancer is a hematologic cancer selected from the group consisting of leukemias, lymphomas, and myelomas.

40. A method as described in embodiment 39, in which the cancer is selected from anaplastic large-cell lymphoma, non-Hodgkin's lymphoma, Hodgkin's lymphoma, B-cell lymphoma, T-cell lymphoma, mantle cell lymphoma, histiocytic lymphoma, T-cell leukemia, chronic lymphocytic leukemia, multiple myeloma, chronic myelogenous leukemia, acute lymphocytic (lymphoblastic) leukemia, acute myelogenous leukemia, acute myeloblastic leukemia, and plasma cell leukemia.

41. A method as described in any of embodiments 38-40, in which the dosing regimen comprises administering the composition to the patient once a week with a rest period of 6 days between each administration.

42. A method as described in any of embodiments 38-41, in which the dosing regimen comprises administering the composition to the patient to provide a dose of about 1 to about 500 mg of the active compound.

43. A method as described in any of embodiments 38-41, in which the dosing regimen comprises administering the composition to the patient to provide a dose of about 10 to about 200 mg of the active compound.

44. A solid dispersion extrudate, comprising an active compound selected from:

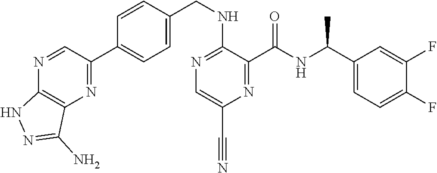 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide,

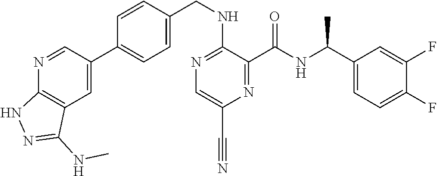 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and

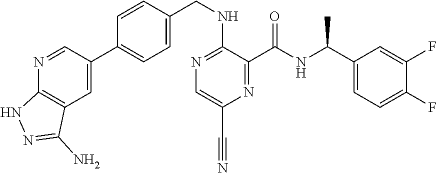 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide;
   the extrudate further comprising copovidone, PEG 1500, and d-α-tocopheryl polyethylene glycol 1000 succinate.

45. A solid dispersion extrudate as described in embodiment 44, comprising:

(a) about 5% to about 15% w/w of the active compound;

(b) about 10% to about 20% w/w of PEG 1500;

(c) about 60% to about 80% w/w of copovidone; and

(d) about 5% to about 15% w/w of d-α-tocopheryl polyethylene glycol 1000 succinate.

46. A solid dispersion extrudate as described in embodiment 45, comprising about 10% w/w of the active compound.

47. A solid dispersion extrudate as described in embodiment 45, comprising about 10% w/w of PEG 1500.

48. A solid dispersion extrudate as described in embodiment 45, comprising about 70% w/w of copovidone.

49. A solid dispersion extrudate as described in embodiment 45, comprising about 10% w/w of d-α-tocopheryl polyethylene glycol 1000 succinate.

50. A pharmaceutical composition for use in treating cancer in a patient, in which the growth, proliferation, or survival of the cancer is dependent on a PDK1-PIF-mediated substrate interaction, comprising (a) the solid dispersion extrudate as described in any of embodiments 44-49, and (b) a pharmaceutically acceptable carrier.

51. A pharmaceutical composition for use in a combinational therapy of treating cancer in a patient, comprising (a) the solid dispersion extrudate as described in any of embodiments 44-49, and (b) a pharmaceutically acceptable carrier, in which the combinational therapy further comprises an effective amount of a second anti-cancer agent.

52. A method of treating cancer in a patient in which the growth, proliferation, or survival of the cancer is mediated by PDK1 activity, comprising administering to said subject a therapeutically effective amount of a pharmaceutical composition as described in any of embodiments 1-25, 50, or 51, or an orally administrable preparation as described in any of embodiments 26-31.

53. A method of treating cancer in a subject in need thereof by inhibiting PDK1-PIF mediated substrate interaction-dependent cancer cell growth, proliferation, or survival, comprising administering to said subject a therapeutically effective amount of a pharmaceutical composition as described in any of embodiments 1-25, 50, or 51, or an orally administrable preparation as described in any of embodiments 26-31.

54. A method for inhibiting the growth, proliferation, or survival of cancer cells by inhibiting Akt-independent cancer cell growth, proliferation, or survival pathways dependent on PDK1-PIF mediated substrate interaction, the method comprising contacting the cancer cells with an effective amount of a pharmaceutical composition as described in any of embodiments 1-25, 50, or 51, or an orally administrable preparation as described in any of embodiments 26-31.

55. A method of inhibiting the growth, proliferation, or survival of cancer cells, the growth, proliferation, or survival of which is dependent on PIF-mediated substrate binding by PDK1, the method comprising contacting the cancer cells with a pharmaceutical composition as described in any of embodiments 1-25, 50, or 51, or an orally administrable preparation as described in any of embodiments 26-31, in an amount sufficient to inhibit growth, proliferation, or survival of the cancer cells.

56. A method of inhibiting the growth, proliferation, or survival of cancer cells in which PDK1-PIF-mediated substrate interaction-dependent cell survival pathways are implicated, comprising contacting the cells with a pharmaceutical composition as described in any of embodiments 1-25, 50, or 51, or an orally administrable preparation as described in any of embodiments 26-31, whereby growth, proliferation, or survival of the cancer cells is inhibited.

57. A method of treating cancer in a subject in need thereof by inhibiting RSK2-dependent cancer cell growth, proliferation or survival, comprising administering to said subject a therapeutically effective amount of a pharmaceutical composition as described in any of embodiments 1-25, 50, or 51, or an orally administrable preparation as described in any of embodiments 26-31.

58. A method of treating cancer in a subject in need thereof by inhibiting Akt-independent cancer cell growth, proliferation, or survival, comprising administering to said subject a therapeutically effective amount of a pharmaceutical composition as described in any of embodiments 1-25, 50, or 51, or an orally administrable preparation as described in any of embodiments 26-31.

59. The method of any one of embodiments 52-58, in which the active compound in the composition or preparation is:

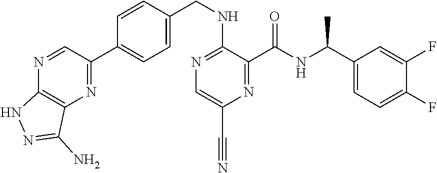 [SMILES: C[C@H](NC(=O)c1nc(C#N)cnc1NCc1ccc(-c2cnc3[nH]nc(N)c3n2)cc1)c1ccc(F)c(F)c1]

3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide or a pharmaceutically acceptable salt thereof.

60. The method of any one of embodiments 52-58, in which the active compound in the composition or preparation is:

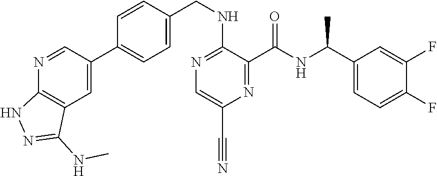 [SMILES: CNc1n[nH]c2ncc(-c3ccc(CNc4ncc(C#N)nc4C(=O)N[C@@H](C)c4ccc(F)c(F)c4)cc3)cc12]

6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide or a pharmaceutically acceptable salt thereof.

61. The method of any one of embodiments 52-58, in which the active compound in the composition or preparation is:

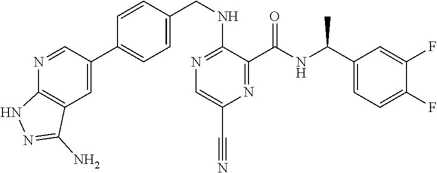 [SMILES: C[C@H](NC(=O)c1nc(C#N)cnc1NCc1ccc(-c2cnc3[nH]nc(N)c3c2)cc1)c1ccc(F)c(F)c1]

3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide or a pharmaceutically acceptable salt thereof.

62. The method of any one of embodiments 52-58, in which the cancer is a hematologic cancer selected from the group consisting of leukemias, lymphomas, and myelomas.

63. The method of embodiment 62, in which the hematologic cancer is selected from anaplastic large-cell lymphoma, non-Hodgkin's lymphoma, Hodgkin's lymphoma, B-cell lymphoma, T-cell lymphoma, mantle cell lymphoma, histiocytic lymphoma, T-cell leukemia, chronic lymphocytic leukemia, multiple myeloma, chronic myelogenous leukemia, acute lymphocytic (lymphoblastic) leukemia, acute myelogenous leukemia, acute myeloblastic leukemia, and plasma cell leukemia.

64. Use of a pharmaceutical composition as described in in any of embodiments 1-25, 50, or 51, in the preparation of a medicament for use in the treatment of cancer in which the growth, proliferation, or survival of the cancer is dependent on a PDK1-PIF-mediated substrate interaction.

65. Use of a container and a medicament for the treatment of cancer in which the growth, proliferation, or survival of the cancer is dependent on a PDK1-PIF-mediated substrate interaction, in which the medicament comprises a solid dispersion extrudate as described in any of embodiments 44-49 and a pharmaceutically acceptable excipient.

While we have described various aspects and embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A pharmaceutical composition comprising a solid dispersion extrudate comprising:
a polymer carrier,
a solubilizer/plasticizer,
a bioavailability enhancer, and
an active compound selected from: 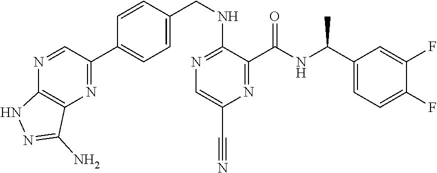 [SMILES: C[C@H](NC(=O)c1nc(C#N)cnc1NCc1ccc(-c2cnc3[nH]nc(N)c3n2)cc1)c1ccc(F)c(F)c1]
3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide, 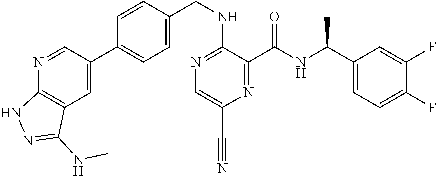 [SMILES: CNc1n[nH]c2ncc(-c3ccc(CNc4ncc(C#N)nc4C(=O)N[C@@H](C)c4ccc(F)c(F)c4)cc3)cc12]
6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and 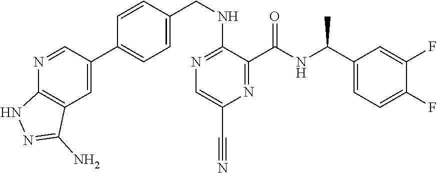 [SMILES: C[C@H](NC(=O)c1nc(C#N)cnc1NCc1ccc(-c2cnc3[nH]nc(N)c3c2)cc1)c1ccc(F)c(F)c1]
3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
or a pharmaceutically acceptable salt thereof.

2. The pharmaceutical composition of claim 1, wherein the polymer carrier is a vinylpyrrolidinone-vinyl acetate copolymer.

3.-5. (canceled)

6. The pharmaceutical composition of claim 1, wherein the solubilizer/plasticizer is PEG 1500.

7.-8. (canceled)

9. The pharmaceutical composition of claim 1, wherein the bioavailability enhancer is d-α-tocopheryl polyethylene glycol 1000 succinate (TPGS).

10.-11. (canceled)

12. The pharmaceutical composition of claim 1, wherein the amount of the active compound in the extrudate is about 5% to about 35% w/w.

13.-17. (canceled)

18. The pharmaceutical composition of claim 1, wherein the solid dispersion extrudate is substantially amorphous, as determined by x-ray powder diffraction analysis.

19.-21. (canceled)

22. The pharmaceutical composition of claim 1, further comprising about 50% to about 90% w/w of one or more pharmaceutically acceptable excipients.

23. An orally administrable preparation of an active compound comprising:
a solid dispersion extrudate comprising:
an active compound,
a polymer carrier,
a solubilizer/plasticizer, and
a bioavailability enhancer; and
one or more pharmaceutically acceptable excipients;
wherein the active compound is selected from: 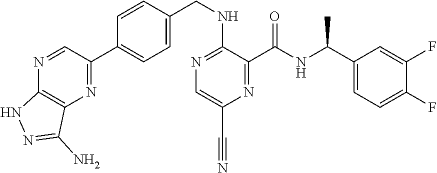 [SMILES: C[C@H](NC(=O)c1nc(C#N)cnc1NCc1ccc(-c2cnc3[nH]nc(N)c3n2)cc1)c1ccc(F)c(F)c1]
3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide, 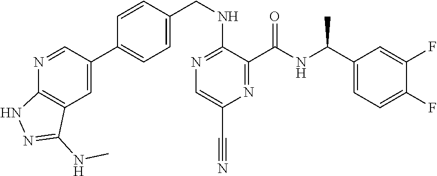 [SMILES: CNc1n[nH]c2ncc(-c3ccc(CNc4ncc(C#N)nc4C(=O)N[C@@H](C)c4ccc(F)c(F)c4)cc3)cc12]
6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and 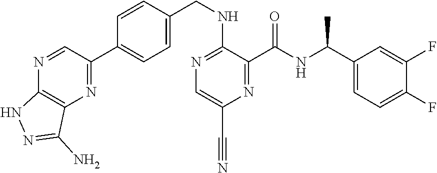 [SMILES: C[C@H](NC(=O)c1nc(C#N)cnc1NCc1ccc(-c2cnc3[nH]nc(N)c3n2)cc1)c1ccc(F)c(F)c1]
3-[4-(3-Amino-1H-pyrazolo[3,4-b]pyridin-5-yl)-benzylamino]-6-cyano-pyrazine-2-carb oxylic acid [1-(3,4-difluoro-phenyl)-ethyl]-amide,
or a pharmaceutically acceptable salt thereof.

24. The pharmaceutical composition of claim 22, wherein the pharmaceutically acceptable excipients are selected from microcrystalline cellulose, pregelatinized starch, magnesium stearate, and combinations thereof.

25.-28. (canceled)

29. A process for preparing a pharmaceutical composition,
which comprises the steps of:
(i) hot melt extruding a mixture of:
a polymer carrier,
a solubilizer/plasticizer,
a bioavailability enhancer, and
an active compound selected from: 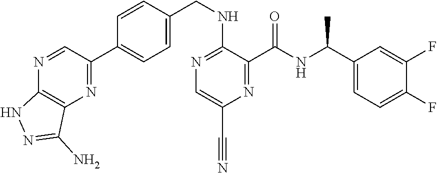 [SMILES: C[C@H](NC(=O)c1nc(C#N)cnc1NCc1ccc(-c2cnc3[nH]nc(N)c3n2)cc1)c1ccc(F)c(F)c1]
3-[4-(3-Amino-1H-pyrazolo[3,4-b]pyrazin-5-yl)-benzylamino]-6-cyano-pyrazine-2-carboxylic acid [1-(3,4-difluoro-phenyl)-ethyl]-amide, 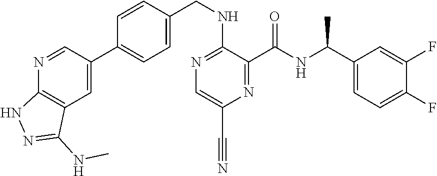 [SMILES: CNc1n[nH]c2ncc(-c3ccc(CNc4ncc(C#N)nc4C(=O)N[C@@H](C)c4ccc(F)c(F)c4)cc3)cc12]
6-Cyano-3-[4-(3-methylamino-1H-pyrazolo[3,4-b]pyridin-5-yl)-benzylamino]-pyrazine-2-carboxylic acid [1-(3,4-difluoro-phenyl)-ethyl]-amide, and 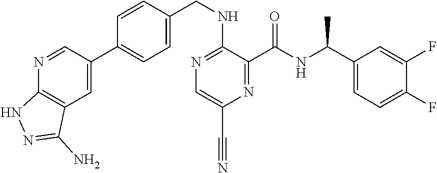 [SMILES: C[C@H](NC(=O)c1nc(C#N)cnc1NCc1ccc(-c2cnc3[nH]nc(N)c3c2)cc1)c1ccc(F)c(F)c1]
3-[4-(3-Amino-1H-pyrazolo[3,4-b]pyridin-5-yl)-benzylamino]-6-cyano-pyrazine-2-carboxylic acid [1-(3,4-difluoro-phenyl)-ethyl]-amide,
or a pharmaceutically acceptable salt thereof,
to form a solid dispersion extrudate; and
(ii) blending the resulting solid dispersion extrudate with one or more pharmaceutically acceptable excipients.

30. The process of claim 29, wherein the polymer carrier is a vinylpyrrolidinone-vinyl acetate copolymer.

31. The process of claim 29, whereinthe solubilizer is PEG 1500.

32. The process of claim 29, wherein the bioavailability enhancer is d-α-tocopheryl polyethylene glycol 1000 succinate.

33. The process of claim 29, wherein the extruding is carried out in an extruder operating with a barrel temperature comprising stages ranging from about 35° C. to about 160° C.

34. The process of claim 29, wherein the extruding is carried out in an extruder operating with a melt temperature ranging from about 95° C. to about 160° C.

35. A method for the treatment of cancer in a patient in need thereof, comprising administering an effective amount of a pharmaceutical composition of claim 1 according to an intermittent dosing regimen, wherein the dosing regimen comprises administering the composition once or twice weekly and wherein the amount of the active moiety administered each week is from about 1 mg to about 500 mg.

36. The method of claim 35, wherein the cancer is a hematologic cancer.

37. The method of claim 36, wherein the hematologic cancer is a leukemia, a lymphoma, or a myeloma.

38.-44. (canceled)

45. The method of claim 35, wherein the cancer is dependent on a PDK1-PIF-mediated substrate interaction.

46. The method of claim 35, wherein the method further comprises a second anti-cancer agent.
